(19) United States
(12) Patent Application Publication
(10) Pub. No.: US 2021/0275290 A1
(43) Pub. Date: Sep. 9, 2021

(54) SOFT SUTURE STAPLE SYSTEM WITH TETHERED ANCHORING MECHANISM

(71) Applicants: Brian J. DOMECUS, San Carlos, CA (US); Scott J. BARON, Menlo Park, CA (US); Michael CHUANG, Tustin, CA (US)

(72) Inventors: Brian J. DOMECUS, San Carlos, CA (US); Scott J. BARON, Menlo Park, CA (US); Michael CHUANG, Tustin, CA (US)

(21) Appl. No.: 17/192,773

(22) Filed: Mar. 4, 2021

Related U.S. Application Data
(60) Provisional application No. 62/985,654, filed on Mar. 5, 2020.
(60) Provisional application No. 63/090,085, filed on Oct. 9, 2020.
(60) Provisional application No. 63/154,489, filed on Feb. 26, 2021.

(51) Int. Cl. A61F 2/08; A61B 17/064; A61B 17/10

(52) U.S. Cl. CPC A61F 2/0811; A61B 17/0642; A61B 2017/0647; A61F 2002/0852; A61F 2002/0888; A61B 17/10

(57) ABSTRACT

Soft suture staple system with tethered anchoring mechanisms are described herein. A tissue anchor assembly may generally comprise an insertion device having a first and second delivery cannula where a first tip and a second tip may each be removably positioned within a respective terminal opening of a delivery cannula. A flexible staple having a body portion, a first leg portion, and a second leg portion may have its leg portions configured to fold proximally within a respective delivery cannula. A tensioning suture may pass through the flexible staple such that application of a tensioning force upon a proximal portion of the tensioning suture may urge the first leg and second leg to each reconfigure into a collapsed, anchoring configuration.

### CROSS-REFERENCE TO RELATED APPLICATIONS

This application claims the benefit of priority to U.S. Prov. 62/985,654 filed Mar. 5, 2020; 63/090,085 filed Oct. 9, 2020; and 63/154,489 filed Feb. 26, 2021, each of which is incorporated herein by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The present disclosure relates generally to suture anchors utilized in a surgical procedure to anchor soft tissue to bone.

### BACKGROUND OF THE INVENTION

Several options are currently available to surgeons treating Partial Articular Surface rotator cuff tears, also known as partial articular supraspinatus tendon avulsion (PASTA) tears. For instance, the tears may be repaired arthroscopically with metal or plastic darts or staples, or with trans-tendinous suture anchors. Alternatively, healthy cuff tissue may be debrided to complete the tear, allowing the surgeon to employ regular full thickness tendinous repair techniques. Some arthroscopists choose to simply decompress and/or debride the surrounding tissues and leave the tear unrepaired.

Each of the above options has disadvantages. Techniques involving metal or plastic staples require second surgeries to remove the staples, and present a risk of displacement or loosening. Suture anchor repairs are time-consuming and technically demanding. Leaving the tear unrepaired leads to persistently poor outcomes. As a result, current methods of PASTA tear repairs have the worst clinical outcomes of all repairable rotator cuff tears.

Another challenging shoulder pathology is anterior inferior instability, which is frequently caused by Hills-Sachs defects in the humeral head after shoulder dislocation. This can be treated using the remplissage procedure, wherein the defect is filled with the infraspinatus tendon and posterior-superior capsule. Traditionally, this is a very difficult procedure involving percutaneous suture anchor placement.

These and other problems associated with current suture devices and techniques are addressed by the present disclosure as discussed below.

### SUMMARY OF THE INVENTION

The present invention relates to a system including an introduction cannula, an insertion device, and a suture anchor formed as a staple with two bone anchoring legs and a bridge between the legs. Other variations may incorporate additional features as desired.

One variation of a cannula may have, e.g., an oval cross-section, with a major diameter and a minor diameter. In other variations, the cannula may be sized to have other cross-sectional shapes, such as a round configuration. The major diameter of the oval cross-sectional shape may be sized to fit the widest dimension of the soft suture staple insertion device while the minor diameter may be sized to fit the shaft portion, e.g., round shaft portion, of the insertion device, and/or other standard round arthroscopic instruments. The cannula may define a lumen for positioning of the soft suture insertion device and may further include a covering or seal which cover the distal opening of the lumen of cannula. The seals may have special cuts or openings in the material of the seal that may match the geometry of the forked tip of the insertion device to provide a good seal and minimal leakage.

The insertion device may be configured to include a forked tip having two delivery cannulas extending in parallel with one another from a transition portion of the insertion device which adapts the delivery cannulas to a larger main shaft of the insertion device. This shaft may also be tubular and the tensioning sutures are passed through this shaft and out of the handle. The delivery cannulas may be shaped in various cross-sectional shapes and their lengths may be varied to be uniform with one another or to have different lengths. Furthermore, one or both of the delivery cannulas may be angled with respect to one another relative to the insertion shaft supporting each of the cannulas. Additionally, the delivery cannulas may be spaced relative to one another at various distances depending upon the desired distance between the anchors to be deployed from the insertion device. Moreover, other variations of the insertion device may include a single cannula or more than two cannulas.

A distal portion of the delivery cannulas may have a respective notch or opening extending lengthwise along a length of the delivery cannulas through which the securement anchors may be deployed. Each of the delivery cannulas may include a piercing tip removably attached to a distal end of the delivery cannulas and also each may be coupled to a respective portion of a suture staple via a tether such as a tether suture.

The soft suture staple itself may be composed of three parts. The main body of the staple may comprise a flexible hollow tubular body with tensioning sutures passed through the lumen for at least a portion of the length. The main tubular body may be shaped such that each leg of the staple extends into each delivery cannula and is folded back upon itself within the cannula.

There are one or more sutures which pass through the lumen of the soft suture staple which serve to both activate the fixation mechanism on each leg of the suture as well as to tension the staple to compress the soft tissue down onto the bone. The tensioning sutures are passed through the lumen of the main tubular body and the suture which exits the end of the tubular body may then be routed back into the lumen of the tubular body through the sidewall of the tubular body. Thus, each leg of the staple forms a loop of the tensioning suture through the folded back portion of the tubular body. When these sutures are tensioned the tubular main body is compressed and pulled into a toroidal shape which creates interference with the narrow hole in the bone.

The soft suture staple includes hard tips located at the distal end of each leg of the staple. These hard tips enable the staple to be advanced directly into bone without the need for pre-punched or pre-drilled holes. These tips are shaped with a sharp point in order to pierce through the soft tissue with minimal tearing or cutting, and also to be durable enough to penetrate the target bone. These tips may be made from a typical implantable metal such as stainless steel, cobalt chromium, titanium, or an implantable hard plastic such as polyetheretherketone (PEEK), or a bioabsorbable material with sufficient hardness such as magnesium or PLLA (polylactic acid), etc.

Alternatively, the hard tips may be connected to the soft suture staple in various ways to improve the fixation of each leg of the staple. In one embodiment the hard tips are tethered to each leg of the staple with a thin suture at anchoring points, or alternatively with a small protruding hook or loop coming off the proximal end of the hard tip. This tether provides a temporary anchoring force on the proximal end of each leg of the staple at respective attachments and this prevents the staple from loosening or backing out of each respective opening while the tensioning sutures are tensioned.

Another embodiment of a soft staple may utilize the insertion device and deployable tips. However, this variation utilizes a tubular body with each leg of the staple which may be held in tension during retraction of the insertion device and which does not begin to compress inside the legs of the delivery cannulas of the insertion device. The staple may have its terminal ends of each leg positioned into proximity of the respective tips rather than having its legs folded back upon itself as described above. The terminal ends of each leg may be temporarily connected, e.g., in the form of a hook feature or a small region that is heat fused to the hard tip.

Tethers may attach along each leg and pass slidingly through tip attachment points and through or along each leg proximally back through the insertion device. The tensioning suture may extend through the insertion device and through the staple and each leg where it may be slidingly attached to either the tips or to the distal ends of each leg.

One variation of a tissue anchor assembly may generally comprise an insertion device having a first delivery cannula and a second delivery cannula adjacent to the first delivery cannula such that the first and second delivery cannulas project distally while defining a notch or opening along each delivery cannula, a first tip removably positioned within a first terminal opening of the first delivery cannula and a second tip removably positioned within a second terminal opening of the second delivery cannula, wherein each of the first and second tips is configured to pierce a tissue region, a flexible staple having a body portion and a first leg portion at a first end of the body portion and a second leg portion at a second end of the body portion, wherein each of the first and second leg portions is configured to fold proximally upon itself within a respective first and second delivery cannula such that the body portion extends between the first and second delivery cannula, a first tether coupling the first tip to the first leg and a second tether coupling the second tip to the second leg, and a tensioning suture passing through the flexible staple such that application of a tensioning force upon a proximal portion of the tensioning suture urges the first leg and second leg to each reconfigure into a collapsed, anchoring configuration.

One method of securing a tissue anchor may generally comprise advancing a first delivery cannula and a second delivery cannula located adjacent to the first delivery cannula directly into a tissue region such that a first channel and a second channel are formed respectively within the tissue region, retracting the first delivery cannula and the second delivery cannula from the first channel and the second channel such that a first tip is detached from the first delivery cannula and remains within the first channel and a second tip is detached from the second delivery cannula and remains within the second channel, deploying a first leg portion from the first cannula and a second leg portion from the second cannula such that the first leg portion and second leg portion remain connected to one another via a body portion of a flexible staple, wherein the first leg portion is folded proximally upon itself within the first channel and the second leg portion is folded proximally upon itself within the second channel, and wherein the first leg portion is coupled to the first tip within the first channel via a first tether and the second leg portion is coupled to the second tip within the second channel via a second tether, and applying a force to a tensioning suture passing through the body portion, the first leg, and the second leg such the first leg portion and the second leg portion are each urged to reconfigure into a collapsed, anchoring configuration within the respective first and second channels.

Yet another variation of a tissue anchor assembly may generally comprise a first tip and a second tip each configured to pierce a tissue region, a flexible staple having a body portion and a first leg portion at a first end of the body portion and a second leg portion at a second end of the body portion, wherein each of the first and second leg portions is configured to fold proximally upon itself, a first tether coupling the first tip to the first leg and a second tether coupling the second tip to the second leg, and a tensioning suture passing through the flexible staple such that application of a tensioning force upon a proximal portion of the tensioning suture urges the first leg and second leg to each reconfigure into a collapsed, anchoring configuration.

Yet another variation of a tissue anchor assembly may generally comprise an insertion device having a first delivery cannula and a second delivery cannula adjacent to the first delivery cannula such that the first and second delivery cannulas project distally while defining a notch or opening along each delivery cannula, a first tip removably positioned within a first terminal opening of the first delivery cannula and a second tip removably positioned within a second terminal opening of the second delivery cannula, wherein each of the first and second tips is configured to pierce a tissue region, a flexible staple having a body portion and a first leg portion at a first end of the body portion and a second leg portion at a second end of the body portion, a first tether coupling the first tip to the first leg and a second tether coupling the second tip to the second leg, and a tensioning suture passing through the flexible staple such that application of a tensioning force upon a proximal portion of the tensioning suture urges the first leg and second leg to each reconfigure into a collapsed, intussuscepted configuration.

Yet another variation of a method of securing a tissue anchor may generally comprise advancing a first delivery cannula and a second delivery cannula located adjacent to the first delivery cannula directly into a tissue region such that a first channel and a second channel are formed respectively within the tissue region, retracting the first delivery cannula and the second delivery cannula from the first channel and the second channel such that a first tip is detached from the first delivery cannula and remains within the first channel and a second tip is detached from the second delivery cannula and remains within the second channel, deploying a first leg portion from the first cannula and a second leg portion from the second cannula such that the first leg portion and second leg portion remain connected to one another via a body portion of a flexible staple, wherein the first leg portion is coupled to the first tip within the first channel via a first tether and the second leg portion is coupled to the second tip within the second channel via a second tether, and applying a force to a tensioning suture passing through the body portion, the first leg, and the second leg such the first leg portion and the second leg portion are each urged to reconfigure into a collapsed, intussuscepted configuration within the respective first and second channels.

Yet another variation of a tissue anchor assembly may generally comprise an insertion device having a delivery cannula which projects distally, a tip removably positioned within a terminal opening of the delivery cannula, wherein the tip is configured to pierce a tissue region, a flexible anchor having a leg portion wherein the leg portion is configured to fold upon itself within the delivery cannula, a tether coupling the tip to the flexible anchor, and a tensioning suture passing through a lumen defined by the flexible anchor such that the suture passes through the flexible anchor to form a cross-over pattern whereby ends of the suture extend through terminal openings of the flexible anchor in opposing directions such that application of a tensioning force upon the tensioning suture urges the flexible anchor to reconfigure into a collapsed, anchoring configuration.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate a perspective and end view of one variation of a cannula having, e.g., an oval cross-section.

FIG. 2A illustrates a side view of one variation of the insertion device which is positioned within the lumen of the cannula.

FIG. 2B illustrates a perspective view of one variation of a deployable punching anchor tip.

FIGS. 2C to 2N illustrate various alternative punching anchor tip embodiments for the anchor system.

FIG. 3A shows a detailed side view illustrating one variation of a single tip deployed within the bone and coupled to a single leg of the suture staple.

FIG. 3B shows a side view of a tip incorporating a tether post and hook.

FIG. 3C shows a side view of a tip incorporating a tether post and loop.

FIGS. 4A to 4C illustrate side views of one soft suture staple anchor variation illustrating how the soft suture staple may be deployed into a region of bone.

FIG. 5 illustrates a side view of another variation of the insertion device pre-loaded with one soft suture anchor embodiment.

FIGS. 6A to 6C illustrate another variation for delivering and deploying the soft staple.

FIGS. 7A and 7B illustrate side views of another variation of a punching tip having a tubular portion with interconnecting portions between two or more radially expanding arm members.

FIG. 7C illustrates a side view of another variation of a punching tip having a tubular portion.

FIG. 7D illustrates a side view of another variation of a punching tip having a tubular portion with radially expanding arm members.

FIGS. 8A to 8E illustrate one variation of a knotless soft suture staple anchor embodiment.

FIGS. 8F-1 to 8F-3 illustrate one example of how a self-locking region of a suture bridge may be implemented.

FIG. 9A shows another variation of a knotless soft suture staple anchoring system.

FIGS. 9B and 9C show yet another variation of a knotless suture tightening assembly where each end of the suture may be formed into the individual legs and reintroduced into two separate spliced sections.

FIGS. 10A to 10C illustrate schematic views of one example for how the spliced portions of suture may be formed.

FIG. 11 shows a side view of one variation of the expandable portion of a suture staple anchor in a flattened configuration to illustrate where openings may be formed along its length.

FIGS. 12A and 12B show another variation of the soft suture staple and deployment in which the tips may be coupled directly to the tensioning suture without the legs of the suture staple being looped.

FIGS. 12C and 12D show yet another variation in which the tensioning suture may incorporate one or more splice sections along the tensioning suture in one or both members of the suture staple.

FIG. 13A shows a variation of a single leg anchoring portion of suture staple formed as a dual loop anchor.

FIGS. 13B and 13C show perspective and bottom views of another variation having a tri-loop suture staple embodiment.

FIGS. 14A to 14E show another variation of an anchoring portion of suture staple in which the anchoring portion of the suture staple may be formed in a looped configuration.

FIGS. 15A to 15C illustrate partial cross-sectional side views of another variation of the anchoring assembly.

FIG. 16 illustrates a partial cross-sectional side view of the anchoring assembly illustrating one variation of a pushrod for facilitating deployment of the staple.

FIGS. 17A to 17C illustrate partial cross-sectional side views of another variation of the anchoring assembly.

FIG. 18 illustrates a partial cross-sectional assembly view of one variation incorporating a bridge pushing mechanism.

FIGS. 19A and 19B illustrate another variation of the insertion device which may be configured for reducing or eliminating anchor pull back during inserter removal.

FIG. 20 shows a side view of another variation of the insertion device where a first delivery cannula may be relatively shorter than a second delivery cannula by some nominal distance.

FIG. 21 shows a side view of another variation of the insertion device in which the delivery cannulas may be angled inwardly relative to a longitudinal axis of the insertion device at some offset angle.

FIGS. 22A and 22B illustrate another variation of the suture staple utilizing a single cannula to deploy a single leg of the suture staple.

FIG. 23 is a diagrammatic view showing another variation of a soft suture staple system.

FIG. 24 is a side view of another variation of the deployment instrument.

FIG. 25 is a partial cross-sectional side view of the deployment instrument of FIG. 24.

FIG. 26 is a detail view of the delivery cannulas of the deployment instrument of FIG. 24.

FIG. 27 is a detail perspective view of the delivery cannulas of the deployment instrument of FIG. 24.

FIG. 28 is a detail perspective view of a partial delivery cannula.

FIG. 29 is a detail partial cross-sectional side view of the delivery cannula and suture staple positioned within prior to deployment.

FIG. 30 is a side view of the suture staple after deployment when seated within the tissue and bone.

FIG. 31 illustrates a perspective view of an optional system for reinforcing a repair made with the suture staple.

FIGS. 32A and 32B illustrate detail side views of another variation of a single leg of the suture staple prior to and after deployment.

FIGS. 33A to 33C show yet another variation of the suture staple incorporating a locking mechanism for the suture staple.

### DETAILED DESCRIPTION OF THE INVENTION

To improve fixation of soft tissue to bone a soft suture staple system has been developed which employs relatively hard tips enabling direct application through soft tissue and bone without requiring pre-punching or pre-drilling holes. This system is particularly useful in situations where the soft tissue to be fixated is still partly intact and covering the underlying bone. In this situation it is desirable to minimize the damage to the remaining soft tissue, and multiple punctures with drills, awls, and traditional anchors generate excessive tissue injury. The soft suture staple system is able to achieve two points of fixation through the soft tissue while only puncturing through the soft tissue one time. Although described in examples as puncturing into bone such as cortical bone, the devices described may be used for securement or attachment into various tissue types and are not limited to securement within bone.

The soft suture staple system may be generally comprised in one variation of a specialized cannula, an insertion device, and the soft staple implants. Other variations may incorporate additional features as desired.

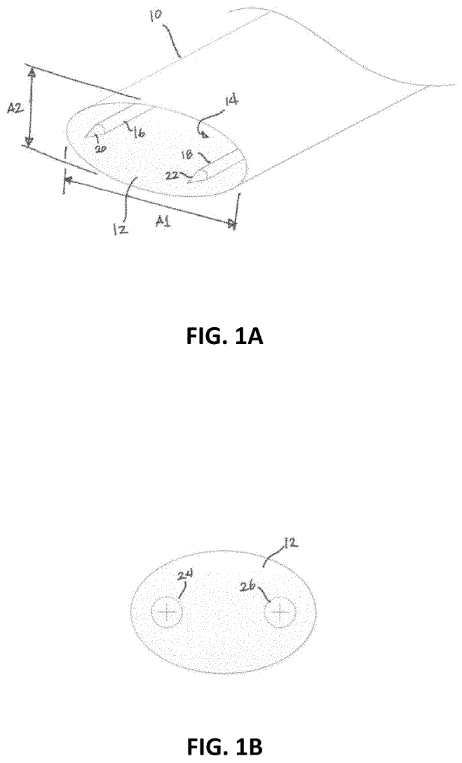

FIG. 1A shows a perspective view of one variation of a cannula 10 having, e.g., an oval cross-section, with a major diameter A1 and a minor diameter A2. In other variations, the cannula may be sized to have other cross-sectional shapes, such as a round configuration. The major diameter A1 of the oval cross-sectional shape may be sized to fit the widest dimension of the soft suture insertion device (as described herein) while the minor diameter A2 may be sized to fit the shaft portion, e.g., round shaft portion, of the insertion device, and/or other standard round arthroscopic instruments. The cannula 10 may define a lumen 14 for positioning of the soft suture insertion device and may further include a covering or seal 12, e.g., two seals, which cover the distal opening of the lumen 14 of cannula 10. The seals 12 may have special cuts or openings 24, 26 in the material of the seal 12 that may match the geometry of the forked tip of the insertion device to provide a good seal and minimal leakage, as shown in the end view of cannula 10 in FIG. 1B.

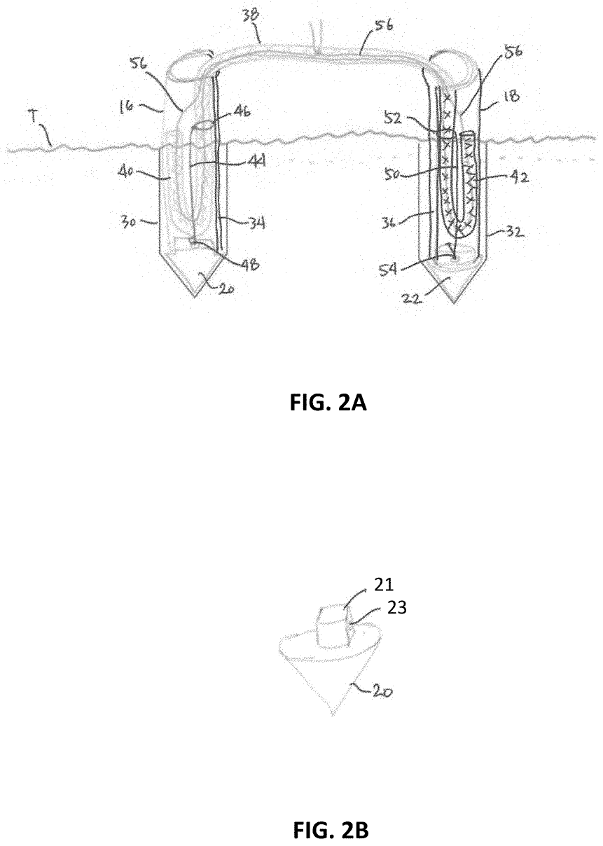

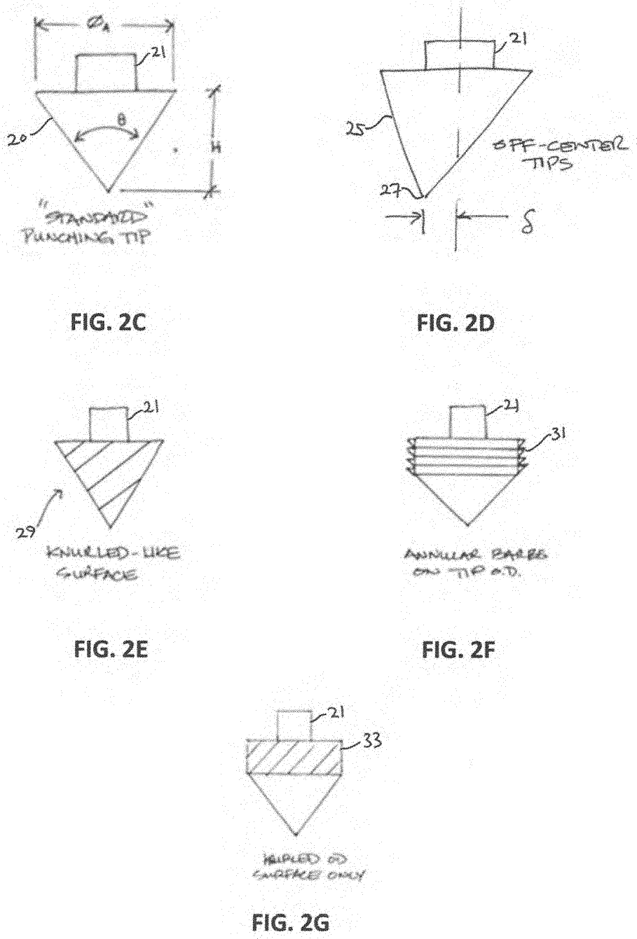

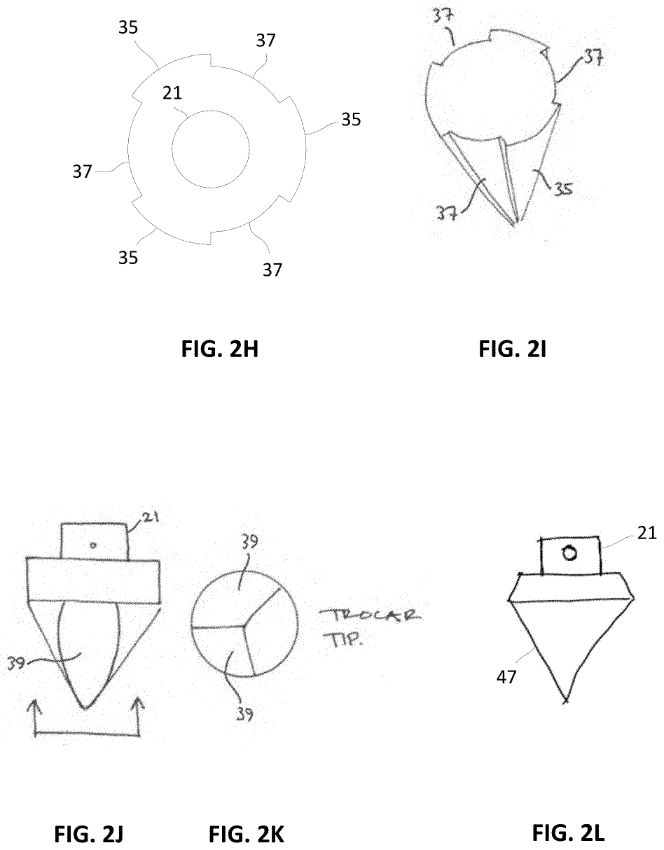

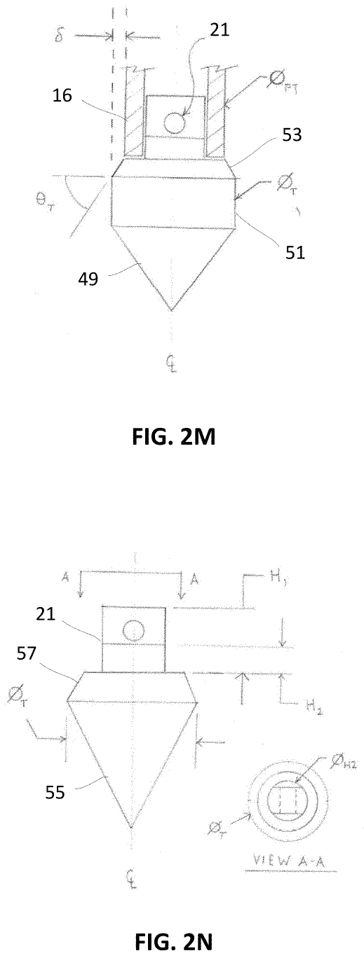

A side view of one variation of the insertion device which is positioned within the lumen 14 of cannula 10 is shown in FIG. 2A. The insertion device may be configured to include a forked tip having two delivery cannulas 16, 18 extending in parallel with respect to one another from the transition portion of the insertion device (not shown) which adapts the delivery cannulas 16, 18 to a larger main shaft of the insertion device. This shaft may also be tubular and the tensioning sutures are passed through this shaft and out of the handle (described in further detail below).

The delivery cannulas 16, 18 may be shaped in various cross-sectional shapes and their lengths may be varied to be uniform with one another or to have different lengths. Furthermore, the delivery cannulas 16, 18 are shown to extend in parallel, but one or both of the delivery cannulas 16, 18 may be angled with respect to one another relative to the insertion shaft supporting each of the cannulas 16, 18. Additionally, the delivery cannulas 16, 18 may be spaced relative to one another at various distances depending upon the desired distance between the anchors to be deployed from the insertion device. Moreover, while two delivery cannulas 16, 18 are shown adjacent to one another, other variations of the insertion device may include a single cannula or more than two cannulas.

A distal portion of the delivery cannulas 16, 18 are shown for clarity purposes and each of the delivery cannulas 16, 18 is shown to have a respective notch or opening 34, 36 extending lengthwise along a length of the delivery cannulas 16, 18 through which the securement anchors and body of the staple 38 may be deployed. The notch or opening 34, 36 may be positioned along each delivery cannula 16, 18 so that they are located opposite to one another to facilitate deployment of the staple from the delivery cannulas 16, 18 (as described in further detail below) although in other variations, the notch or openings 34, 36 may be located at other locations. Each delivery cannulas 16, 18 may include a piercing tip 20, 22 removably attached to a distal end of the delivery cannulas 16, 18 and also each coupled to a respective portion of a suture staple via a tether 44, 50 such as a tether suture.

The insertion device may be sufficiently rigid to be advanced (e.g., malleted by a surgical mallet) into a region of soft tissue and/or bone for deploying the soft suture staple, i.e., the delivery cannulas may each have a column strength sufficient to support being advanced directly the region of soft tissue and/or bone without the need for a predrilled channel. The wall thickness of the delivery cannulas 16, 18 may provide adequate surface area to transfer the advancement forces to the tips 20, 22 while still maintaining a large enough lumen to store the fixation portion of the soft suture staple. This wall thickness may be greater than, e.g., 0.005 in., and less than, e.g., 0.030 in.

The soft suture staple 38 itself may be composed of three parts. The main body of the staple 38 may comprise a flexible hollow tubular body with tensioning sutures passed through the lumen for at least a portion of the length. In other embodiments, the body portion of the staple may be sectioned into 2 or more sections as described in further detail below. In the variation shown in FIG. 2A, the main tubular body may be shaped such that each leg 40, 42 of the staple 38 extends into each delivery cannula 16, 18 and is folded back upon itself within the cannula 16, 18 to form a loop. In other embodiments the main tubular body forms the inverted “U” shape of a staple. The soft suture staple 38 may be comprised of a number of flexible materials but in one variation, the staple 38 may be formed as a braided tubular structure which forms a flexible staple which defines a lumen throughout the length of the staple 38.

There are one or more sutures 56 which pass through the lumen of the soft suture staple 38 which serve to both activate the fixation mechanism on each leg 40, 42 of the suture 38 as well as to tension the staple to compress the soft tissue down onto the bone T. The tensioning sutures 56 are passed through the lumen of the main tubular body and the suture 56 which exits the end of the tubular body may then be routed back into the lumen of the tubular body through the sidewall of the tubular body. Thus, each leg 40, 42 of the staple forms a loop of the tensioning suture 56 through the folded back portion of the tubular body. When these sutures 56 are tensioned the tubular main body 38 is compressed and pulled into a toroidal shape which creates interference with the narrow hole in the bone.

The soft suture staple includes hard tips 20, 22 located at the distal end of each leg 40, 42 of the staple. These hard tips 20, 22 may be removably positioned at the distal end of a respective delivery cannula which is part of a deployment tool, as described below in further detail. The tips 20, 22 may enable the staple 38 to be advanced directly into bone (e.g., malleted, punched, etc.) without the need for pre-punched or pre-drilled holes such that the tips 20, 22 may be advanced into the bone directly and then deployed to remain within the bone once the deployment tool is withdrawn proximally, as described in further detail herein. These tips 20, 22 are shaped with a sharp point in order to pierce through the soft tissue with minimal tearing or cutting, and also to be durable enough to penetrate the target bone. These tips 20, 22 may be made from a typical implantable metal such as stainless steel, cobalt chromium, titanium, or an implantable hard plastic such as polyetheretherketone (PEEK), or a bioabsorbable material with sufficient hardness such as magnesium or PLLA (polylactic acid), etc. The hard tips 20, 22 may be unconnected from the soft suture staple 38 and only provide the required sharp leading edge which enables the staple to be inserted into the bone at the same time as the holes in the bone are formed. A perspective view of a tip 20 is shown in FIG. 2B which illustrates the tapered tip 20 having an anchoring portion 21 located along a proximal portion of the tip 20 and further defining an opening 23 through the anchoring portion 21 for securement of the suture 38 during deployment.

Alternatively, the hard tips 20, 22 may be connected to the soft suture staple 38 in various ways to improve the fixation of each leg of the staple. In one embodiment the hard tips are tethered to each leg of the staple with a thin suture tether 44, 50 at anchoring points 48, 54, or alternatively with a small protruding hook coming off the proximal end of the hard tip. This tether 44, 50 provides a temporary anchoring force on the proximal end of each leg 40, 42 of the staple 38 at respective attachments 46, 52 and this prevents the staple 38 from loosening or backing out of each respective opening 30, 32 while the tensioning sutures 56 are tensioned.

FIGS. 2C to 2L illustrate various alternative tip embodiments for the anchor system. In each of these variations, the tip may be designed to be coupled to both the anchor itself and/or mounted onto the insertion tool.

FIG. 2C shows a side view of the tip 20 having the anchoring portion 21 to illustrate details of a conical-shaped tip which may taper at a predetermined angle Θ from its punching tip to a relatively larger predetermined diameter Ø such that the tip 20 has a height H which are optimized for punching through bone. For each tip embodiment, the outer diameter is optimized such that the outer diameter may be, e.g., 0 to 0.020 in larger than the outer diameter of the fork leg. This difference in diameter on each delivery cannula 16, 18 is optimized to promote contact between the bone and the hard tips 20, 22, to reduce friction between the delivery cannula 16, 18 and the bone for ease of removal, and minimize the punched hole size to maximize anchor fixation in the bone once deployed.

FIG. 2D shows a side view of another variation of the tip 25 in which the piercing tip 27 may be offset δ by an amount relative to a longitudinal axis of the tip such that the tip is formed as an asymmetric conical tip. The offset δ may facilitate the positioning of the piercing tip 27 in positioning the tool when initiating insertion onto a curved bone surface. When used with an insertion device, as described herein, at least two tips 25 may be used where the piercing tip 27 may be positioned in an opposite orientation relative to one another. Reducing the centerline distance between each side of the anchor insertion device may compensate for situations where a curvature of the bone surface may make simultaneous contact between each tip 20, 22 and the bone surface difficult prior to initial insertion into the bone.

FIG. 2E shows a side view of another tip variation where the outer surface of the tip may implement a surface 29 which is roughened or which features a number of projections. For instance, the surface 29 may present a knurled-like surface for enhancing frictional grip between the tip surface and bone surface within the punched hole.

FIG. 2F shows a side view of yet another tip variation in which cylindrical-shape features 31, such as annular barbs or shoulders, may project from an outer surface of the tip. Such features 31 may enable the tip to resist tensioning forces from pulling out the tip from the bone.

FIG. 2G shows a side view of yet another tip variation in which the tip may be configured with a cylindrical surface 33. This surface 33 may similarly present a roughened interface or a surface having a number of projections (e.g., teeth, ribs, barbs, etc.) to enhance the frictional resistance of the tip from being pulled out during insertion device removal (e.g., delivery cannula 16, 18 removal) and during anchor deployment.

FIGS. 2H and 2I show top and perspective views of a partial tip to illustrate a variation in which the conical section of the tip may define cutout portions 37 which culminate or intersect at the piercing tip. The cutout portions 37 may be alternated between portions 35 such that the tip is configured to resist torsional forces when deployed within the bone. Although three cutout portions 37 are illustrated, other variations may incorporate fewer or greater than three cutout portions 37 which may be uniform in dimension or arbitrary depending upon the desired configuration.

FIGS. 2J and 2K show side and bottom views of yet another variation in which the conical portion may be configured to feature a beveled surface 39 similar to a trocar tip. Although three beveled surfaces 39 are shown in this variation, other variations may incorporate two beveled surfaces or greater than three beveled surfaces.

FIG. 2L shows a side view of yet another variation in which the tip may incorporate a coating or covering 47 such as a ceramic or metallic coating. The incorporation of a coating or covering 47 may help to ensure the direct insertion of the tip into and through the bone.

FIG. 2M shows a side view of yet another variation in which the tip 49 may be configured to have an outer diameter which is relatively larger than the outer diameter of the delivery cannula 16 from which the tip 49 is deployed. As shown, the tip 49 may be configured to have a widened portion 51 with an outer diameter Ø_{T} positioned proximally of the tip. The widened portion 51 may reduce in diameter along a tapered portion 53 at some angle Θ_{T} to reduce in diameter such that a shoulder may be formed for abutting against the terminal opening of the delivery cannula 16. Due to the widened portion 51, an annular space δ may be formed between the outer diameter Ø_{T} of widened portion 51 and the outer diameter Ø_{FT} of the delivery cannula 16 such that the anchoring portion 21 is contained within the delivery cannula 16.

FIG. 2N shows a side view and detailed top view of yet another variation where the tip 55 may be configured to have an outer diameter Ø_{T} which is relatively wider than the delivery cannula 16. In this variation, the tip 55 may taper to its widened diameter and then reduce in an opposite taper along a proximal portion 57. The detail view A-A illustrates a top view showing the relative diameter of the widened outer diameter Ø_{T} in comparison to the reduced diameter Ø_{H2} formed by the anchoring portion 21.

Each of the various features shown and described in the tip variations may be applied and used in combination with any of the other features described herein and are intended to be within the scope of the description herein.

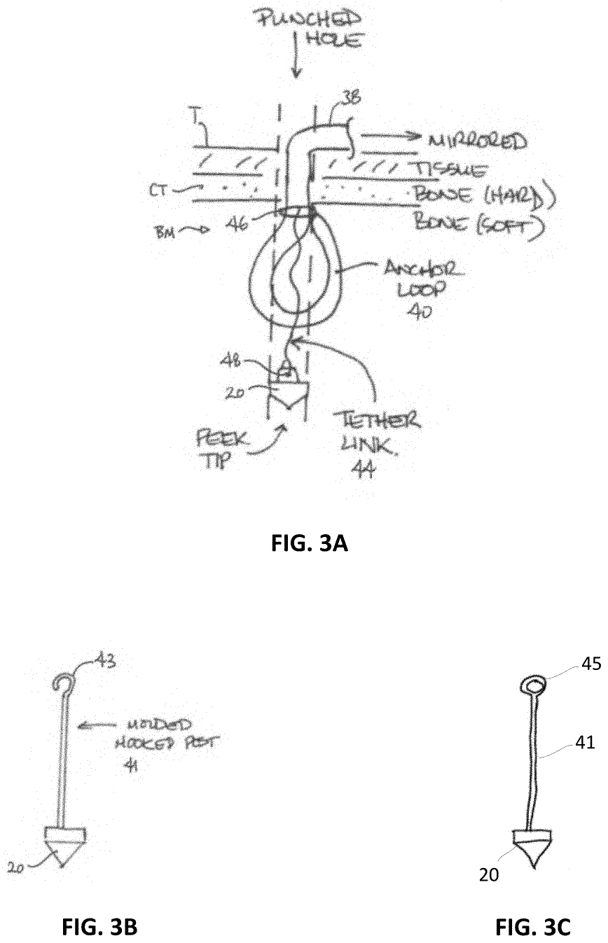

FIG. 3A shows a detailed view illustrating a single tip 20 deployed within the bone and coupled to a single leg of the suture staple 38 (the second leg portion is omitted only for clarity purposes). The tether 44 is shown coupled via attachment point 48 to the tip 20 and extending around a portion a second attachment point 46 to the leg 40 of the suture staple 38. In this example, the various layers through which the tip 20 may be penetrated through and into are shown as including a tissue layer T, the cortical layer CT of the bone which is relatively hard, and the relative softer cancellous layer or bone marrow BM. As the tip 20 is deployed into the bone marrow BM, the leg 40 may expand or reconfigure slightly beyond the constraints of the diameter of the punched insertion hole due to the softer bone marrow BM accommodating the reconfiguration or expansion of leg 40. The tip 20 maintains its position in the bone during the reconfiguration of the leg to maintain the overall anchor position relative to the bone. With the leg 40 reconfigured relative to the opening into the bone, the retention of the staple 38 within the bone once in its anchoring configuration may be facilitated.

As the tip 20 may be fabricated from a number of bioabsorbable materials (or metals), as described herein, the tip 20 may optionally incorporate a post 41 which may be coupled, attached, molded, or otherwise incorporated with the tip 20 to extend proximally for coupling to the suture staple or suture, as shown in the side view of FIG. 3B. At the terminal end of the post 41, an attachment feature 43 such as a hook, hoop, loop, eyelet, etc. may be incorporated or integrated for attachment the to the anchor body. FIG. 3C shows yet another variation where the terminal end of the post 41 may incorporate a closed loop or eyelet 45 instead.

Any of the variations of the tips or coupling features are intended to be integrated in any number of combinations with any of the suture staple variations and deployment instruments as described here.

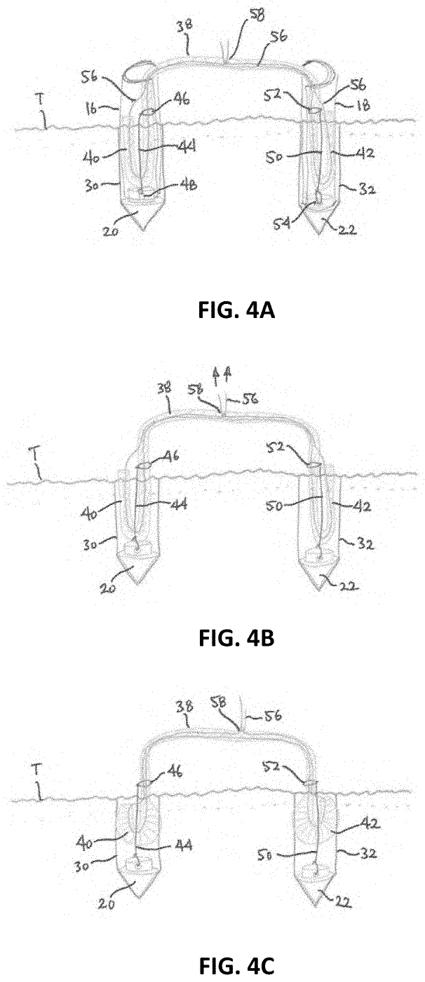

FIGS. 4A to 4C show side views of one variation illustrating how the soft suture staple 38 may be deployed into a region of bone T. FIG. 4A illustrates an example of the delivery cannula 16, 18 having been advanced directly into the region of bone T creating a respective channel 30, 32 within the bone. Once advanced to a desirable depth, the insertion device and delivery cannulas 16, 18 may be retracted proximally allowing for the tips 20, 22 to detach from the respective delivery cannula 16, 18 and remain within the created channel 30, 32. The respective leg 40, 42 of the staple 38 may slide out or pass through the distal opening of the delivery cannulas 16, 18 vacated by the deployment of the tips 20, 22 as well as the respective opening 34, 36 defined along each delivery cannula 16, 18. In order to facilitate deployment, a pushing mechanism (as described in detail below) may force each of the legs 40, 42 and tips 20, 22 to deploy distally from the delivery cannula 16, 18 during placement within the respective channel 30, 32. Once the tips 20, 22 have been distally deployed, the legs 40, 42 may follow distally through the openings at the terminal ends of the delivery cannula 16, 18. The body of the staple 38 may be unrestrained by the insertion device such that it may be deployed distally from the insertion device unhindered such that the body of the staple 38 readily slides through the length of the opening 34, 36 while the legs 40, 42 and tips 20, 22 are deployed with each leg 40, 42 remaining within the respective channel 30, 32, as shown in FIG. 4B. With each leg 40, 42 coupled via a tether 44, 50 to the tip 20, 22, the legs 40, 42 may remain within the channels 30, 32.

The tensioning suture 56 may pass into the length of the soft staple 38 at an insertion point 58. From the insertion point 58, the suture 56 may pass along the body of the staple 38 and through the first leg 40 such that the suture 56 exits out the terminal end of the first leg 40. The suture 56 may then reenter the body of the staple 38 in proximity to the first leg 40 such that it passes back through the body and into the second leg 42 such that the suture exits out the terminal end of the second leg 42. The suture 56 may then reenter the body of the staple 38 in proximity to the second leg 42 such that the suture 56 is passed back into the body and out through insertion point 58 such that the suture may be passed through the entire length of the staple 38 as well as each leg 40, 42. In this manner, the suture 56 may effectively create a continuous looped path through the staple 38 and back out through the insertion device so that the tensioning suture 56 may be tensioned from outside the patient following insertion device removal from the bone. The looped pathway of the suture 56 may maximize the tendency of the folded portions of each leg 40, 42 to compress into their anchoring configuration.

Applying tension to the tensioning suture 56 may pull the ends of each leg 40, 42 into the respective channel 30, 32 to coil upon themselves forming, e.g., a ball shape or a toroidal shape or a coiled configuration, effectively anchoring each end securely within the respective channel 30, 32 and tightening the soft staple 38 against the internal surface of the bone T to retain the feature, such as soft tissue or an ancillary device, to the region of bone, as shown in FIG. 4C. As the tensioning suture 56 is tightened, the tether 44, 50 securely attached between each leg 40, 42 and to the anchored tip 20, 22 may prevent the staple 38 from being pulled proximally out from the bone channels 30, 32.

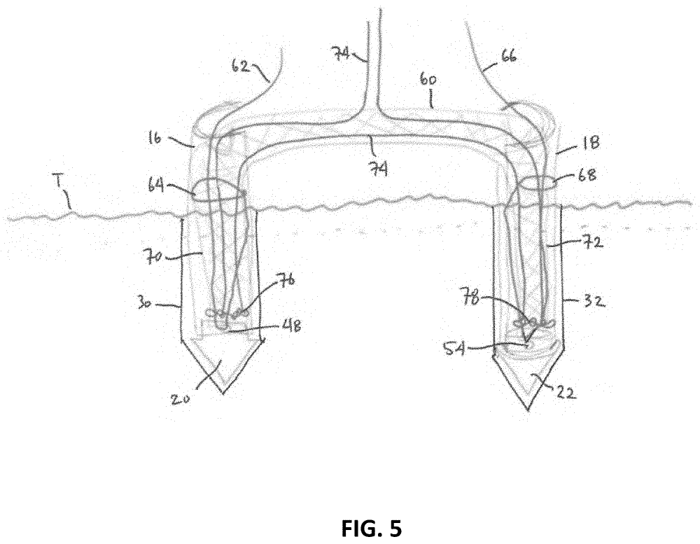

Another embodiment of a soft staple is illustrated in the side view of FIG. 5 which may utilize the insertion device and deployable tips 20, 22. However, this variation utilizes a tubular body with each leg 70, 72 of the staple 60 which may be held in tension during retraction of the insertion device and which does not begin to compress inside the legs of the delivery cannulas 16, 18 of the insertion device. The staple 60 may have its terminal ends of each leg 70, 72 positioned into proximity of the respective tips 20, 22 rather than having its legs folded back upon itself as described above. The terminal ends of each leg 70, 72 may be temporarily connected, e.g., in the form of a hook feature or a small region that is heat fused to the hard tip 20, 22. In the embodiment employing a hook feature, the hook may be sized to provide the temporary tension on the tubular body during retraction of the insertion device, but is able to deform under the force of the tensioning sutures which allows the main body to compress upward toward the surface of the bone.

Tethers 64, 68 may attached along each leg 70, 72 and pass slidingly through tip attachment points 48, 54 and through or along each leg 70, 72 proximally back through the insertion device. The tensioning suture 74 may extend through the insertion device and through the staple 60 and each leg 70, 72 where it may be slidingly attached to either the tips 20, 22 or to the distal ends of each leg 70, 72.

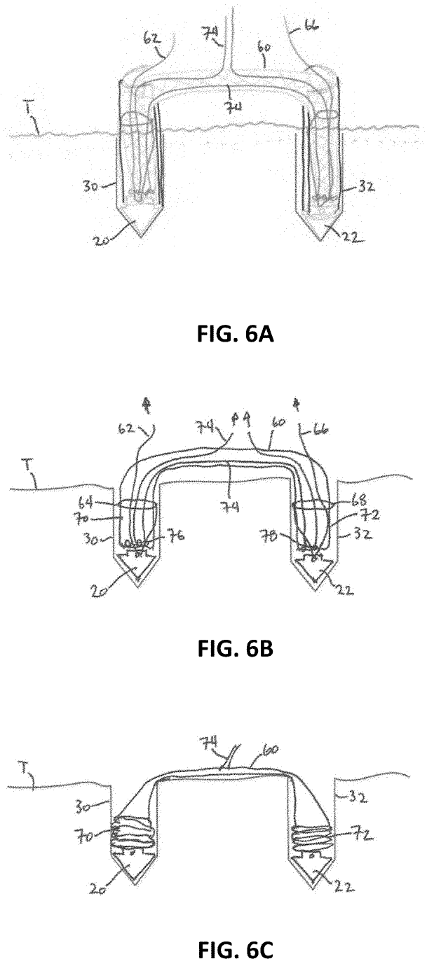

FIGS. 6A to 6C illustrate one variation for delivering and deploying the soft staple 60 where FIG. 6A illustrates how each delivery cannula may be advanced into the tissue T to create the respective channels 30, 32. As the insertion device and delivery cannulas 16, 18 are retracted proximally, the tips 20, 22 may detach and remain in place within the bottom of channels 30, 32 along with the staple 60. As described above, the ends of each leg 70, 72 may pass through the notches defined along the delivery cannulas 16, 18 for deployment into the channels 30, 32, as shown in FIG. 6B. The tethers 62, 66 may be tensioned proximally to pull the legs 70, 72 and the body of staple 60 towards the tips 20, 22 such that the legs 70, 72 are forced to compress and telescope upon itself to form an intussusception which essentially anchors the legs 70, 72 within their respective channels 30, 32, as shown in FIG. 6C. The ends of the tensioning suture 74 may be tensioned proximally to tighten the body of staple 60 and any attached tissue to the staple 60.

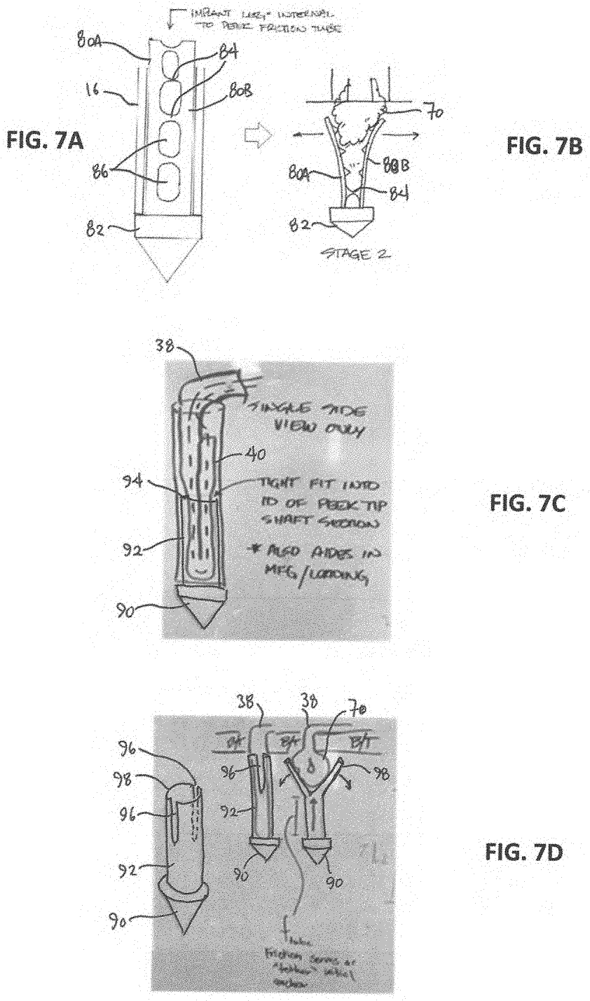

With the deployment of the suture staple described, FIGS. 7A and 7B illustrate side views of alternative punching tip and tether variations which may be used with any of the suture staple variations. The anchoring tip 82 shown may incorporate a cylindrical tube portion two or more arm members 80A, 80B which extend proximally from the tip 82 and which may be joined to one another via interconnecting portions 84 along the length or a portion of the length of the arm members. The suture staple may be positioned within the central lumen of the proximal feature (channel) between arm members 80A, 80B which may maintain a straightened anchor configuration during delivery into and through the bone. The leg of the suture staple may be packed into the channel formed by the arm members 80A, 80B for insertion and delivery into and through the bone. When the leg of the suture staple is reconfigured into its expanded anchoring configuration, as shown by leg 70 in FIG. 7B, the reconfiguration of the leg 70 may selectively break or de-couple one or more of the interconnecting portions 84 such that arm members 80A, 80B may flare radially outwardly away from one another to form an expanded profile which further resists being pulled proximally out from the bone.

Another variation is shown in the side view of FIG. 7C which illustrates another tip 90 having a tubular portion 92 extending from the tip 90 and defining an opening 94 through which the leg 40 of the suture staple 38 may be packed into forming a friction or press fit for delivery and deployment. Yet another variation is shown in the side views of FIG. 7D which illustrates a similar variation of a tip 90 having a tubular portion 92 but where a proximal portion of the tubular portion 92 may define one or more slots or channels 96 which may extend along the portion 92 from the terminal opening. The slots or channels 96 may form arm members 98 which may extend radially away from one another during anchoring of the suture staple 38. As illustrated, once the leg 70 of the suture staple is reconfigured into its anchoring configuration, the force of the expanding leg 70 may press against the arm members 98 such that they deform or reconfigure radially outward into a configuration which further prevents the tip 90 and/or leg 70 from being pulled out proximally from the bone.

As described herein, the tethers may attach along each leg and pass slidingly through tip attachment points and through or along each leg proximally back through the insertion device. The tensioning suture may extend through the insertion device and through the staple and each leg where it may be slidingly attached to either the tips or to the distal ends of each leg. The tethers may be tensioned proximally to pull the legs and the body of staple towards the tips such that the legs are forced to compress and telescope upon itself to form an intussusception which essentially anchors the legs within their respective channels.

However, another variation of the anchoring system may incorporate a knotless staple suture system which may utilize a dual-splice tensionable knotless lock. Such a configuration may be used without the use of a knot pushing mechanism and may allow a single anchor expansion without comprising a second anchor expansion. This variation may also allow for the staple bridging width to vary depending upon the desired length between the anchoring loops.

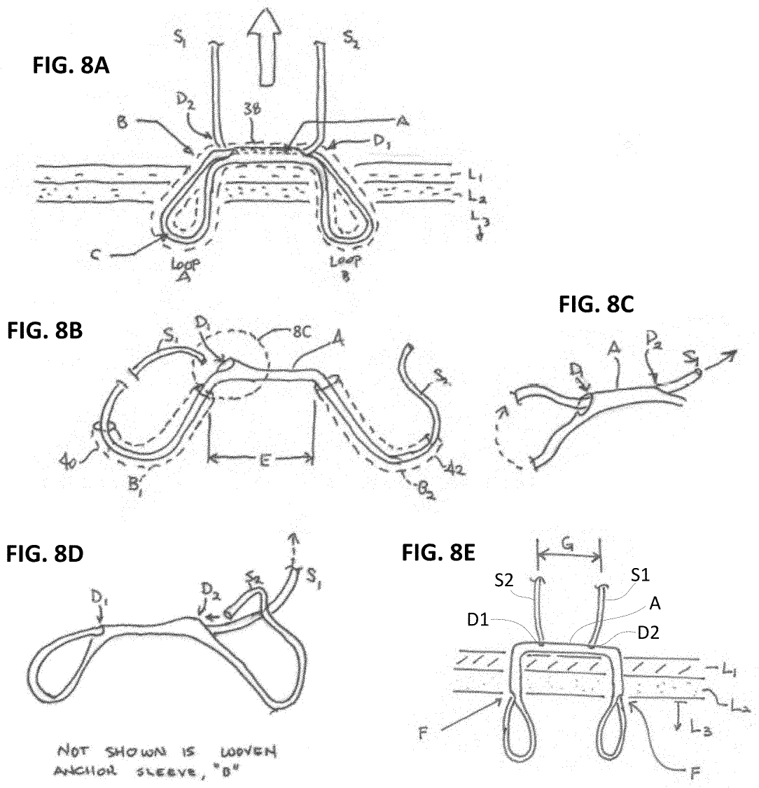

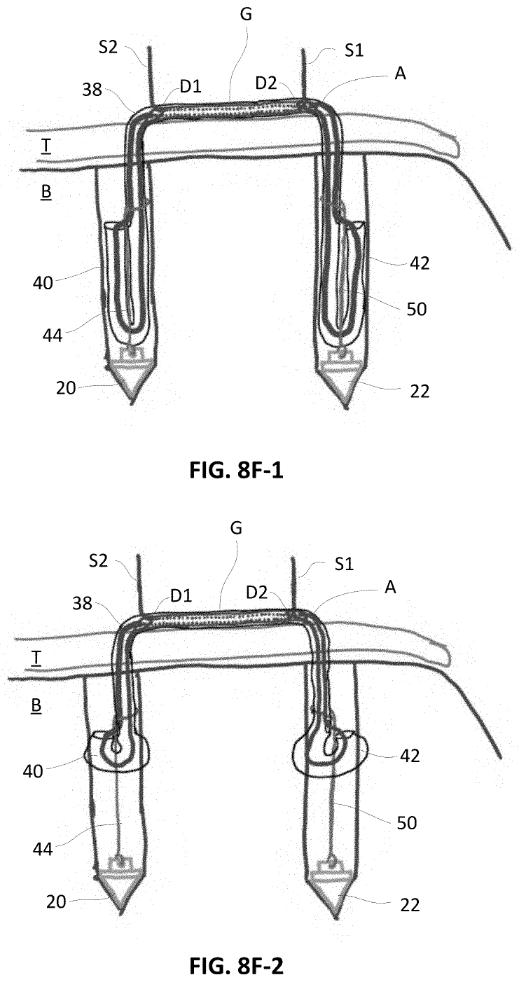

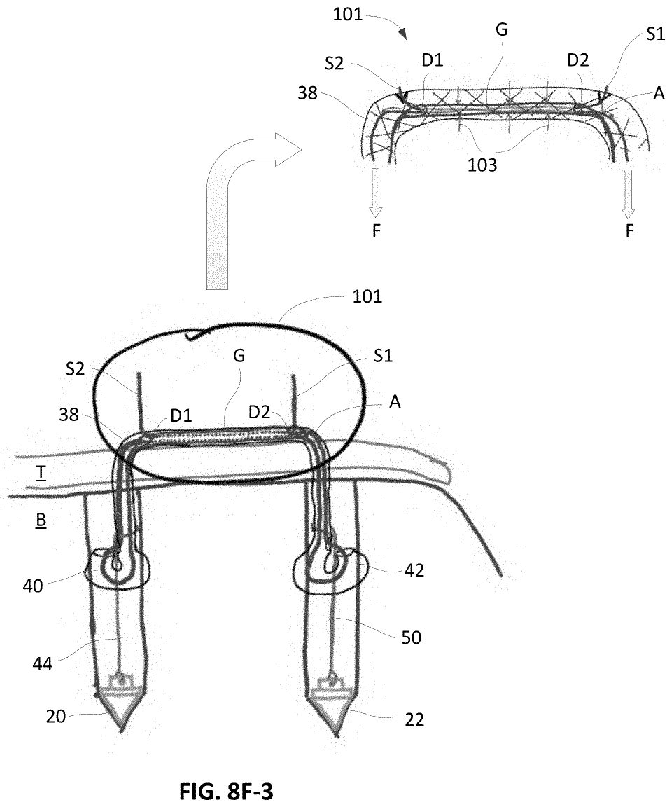

FIGS. 8A to 8E illustrate one variation of a knotless suture anchoring embodiment which may be used with any of the various tip and/or deployment embodiments described herein. FIGS. 8A to 8E illustrate the stepwise manner through which the anchor tensioning suture is routed within the anchor staple body for clarity purposes. These steps have been performed in manufacturing and do not indicate steps to be performed by the user. Further, these figures describe a dual locking embodiment that does not require suture shuttling through the anchor by the user. The tips and other features have been omitted merely for clarity purposes. As shown in FIG. 8A, the assembled knotless configuration is shown where the suture length extends through both legs A, B of the suture staple 38 and is spliced within itself along the bridging portion A such that the opposing terminal ends S1, S2 of the suture length may be extended away from the suture anchor for tightening the suture staple into a knotless anchor. The terminal ends S1, S2 may be trimmed flush to the tissue surface, if desired, once the suture staple 38 has been deployed.

As shown in FIG. 8B, the first terminal end S1 of the suture length may be passed through the first leg 40 of the suture staple 38 such that it exits the suture staple 38 and is introduced back into the suture length between the braided or woven elements forming the suture length at a first point D1. The first terminal end S1 may be passed through a lumen which is defined through the suture itself to then exit from the suture interior lumen between the braided or woven elements forming the suture at a second point D2, as illustrated in the detail view of FIG. 8C. With the first terminal end S1 passed back through the suture from points D1 to D2, the second terminal end S2 of the suture which passes through the second leg 42 of the suture staple 38 may also be introduced into the suture itself along the same portion forming the suture bridge A. In this case, the second terminal end S2 may be passed into the suture at point D2 which functions as an entry point into the suture and then passed out of the suture length through point D1, as illustrated in FIG. 8D. Hence, the first point D1 may function as an entry point into the suture for first terminal end S1 and as an exit point out of the suture for second terminal end S2. Likewise, the second point D2 may function as an exit point out of the suture for first terminal end S1 and as an entry point into the suture for first terminal end S1. In introducing and passing the suture back into itself between the braided or woven elements of the suture, any number of tools may be used such as a splicing horn having a blunt tip which allows for suture to be advanced between braid openings.

In other variations, the terminal ends S1, S2 need not pass into and out of the same points, but may instead be introduced and exited from the suture at different points along the suture length.

When the suture is in a relaxed condition, the braided or woven elements forming the suture may form openings or spacings between through which the first point D1 and second point D2 may be formed. Furthermore, the length E defined between the points D1, D2 may form the length suture bridge A which rests upon the tissue surface between the legs 40, 42 inserted within the bone. Hence, length E may be varied by adjusting the location of points D1 and/or D2 to accordingly increase or decrease the length of the suture bridge E, as needed.

With the loops formed and the suture ends introduced into and out the same suture, tensioning of the suture ends S1, S2 may collapse and cinch down upon itself the braided or woven elements of the suture within the suture bridge A such that the tightened condition of the suture and the formed anchoring ends of the suture staple 38 may be maintained without the use of any knots, as shown in FIG. 8E. With the suture looped back into itself along the suture bridge A, the looped portions of the suture may remain within the openings F formed within the bone by the tips such that the loops reside under the cortical bone layer (or at the target depth in the anchoring tissue). The portion of the suture bridge where the suture ends S1, S2 cross one another and overlap before exiting through their respective entry/exit points D1, D2 may form a self-locking region G. When the suture loops are tensioned, e.g., one at a time or both simultaneously, a first suture within the self-locking region G of bridge A may slip in the direction of applied tension because the direction of applied tension compresses the length of bridge A and opens the core in which it resides. Once the tensioning force is removed, the tension due to the compressed tissue is in the opposite direction which elongates and closes the core of bridge A clamping down upon the two legs of the tension suture within. Similarly, as the second suture leg is tensioned the force again compresses the length of bridge A and opens the core allowing the second tension suture leg to slip in the direction of applied tension. Again once the tensioning force is removed from the second tension suture leg the tension due to compressed tissue acts in the opposite direction which elongates the length of bridge A and closes the core clamping down upon the two legs of the tension suture within. In the fully tensioned state, the self-locking region is resistant to loosening because the two legs of the tension suture are compressed against each other within the self-locking region G and the loosening direction one leg of the tension suture is opposite of the loosening direction of the other leg. If one leg begins to slip within the self-locking region it would tend to drag the other leg in its tightening direction, thus resulting in a net zero change in total tightness. When both legs of the tension suture are tensioned, this spliced self-locking region G (which is separate from the suture staple) may form a self-tightening region which is able to maintain its tightened configuration.

Moreover, the tensioning of the suture may be accomplished with or without the use of a knot pushing mechanism to achieve the desired compression on the tissue. The suture bridge A may allow for movement of the sutures in single direction for each terminal end (e.g., opposite directions for each suture end) of the user applied tension.

FIGS. 8F-1 to 8F-3 illustrate one example of how the self-locking region G of bridge A may be implemented. The suture staple 38 may extend with each end forming a respective leg 40,42 coupled via tether 44, 50 to respective tips 20, 22, as described herein. The suture length having the spliced self-locking region G may be seen extending internally through the length of the suture staple 38 such that the suture may form the loop while extending through the staple 38 such that the legs 40, 42 may be extended back upon itself to also form the suture loop. Each of the suture ends S1, S2 may also be seen as extending back into the suture staple 38 and through the respective entry/exit points D1, D2 along the suture itself to form the self-locking region G, as described.

The configuration in FIG. 8F-1 illustrates the suture staple 38 and suture length after the tips 20, 22 have been introduced through the tissue T and into the bone B and prior to reconfiguring the suture staple 38. FIG. 8F-2 illustrates the suture staple 38 reconfiguring from its deployed profile and into its anchoring (e.g., looped or balled) configuration as the suture length is tensioned. As the suture ends S1, S2 are tensioned, the suture may begin to tighten the legs 40, 42 such that they reconfigure into their collapsed anchoring configurations while the tips 20, 22, anchored within the bone, may prevent the ends of the suture staple 38 from moving upward toward the bone surface during tensioning, via the tethers 44, 50, as previously described.

FIG. 8F-3 shows a detailed view 101 of the self-locking region G to illustrate how the suture bridge A and the spliced self-locking region G may be positioned internally of the suture staple 38 lumen. As the suture is tensioned as indicated by the arrows F, the spliced self-locking region G within the suture staple 38 may form a self-tightening region, as indicated by the direction of compression 103, which is able to maintain its tightened configuration when tension is applied so that the suture staple 38 is also able to maintain its tightened anchoring configuration.

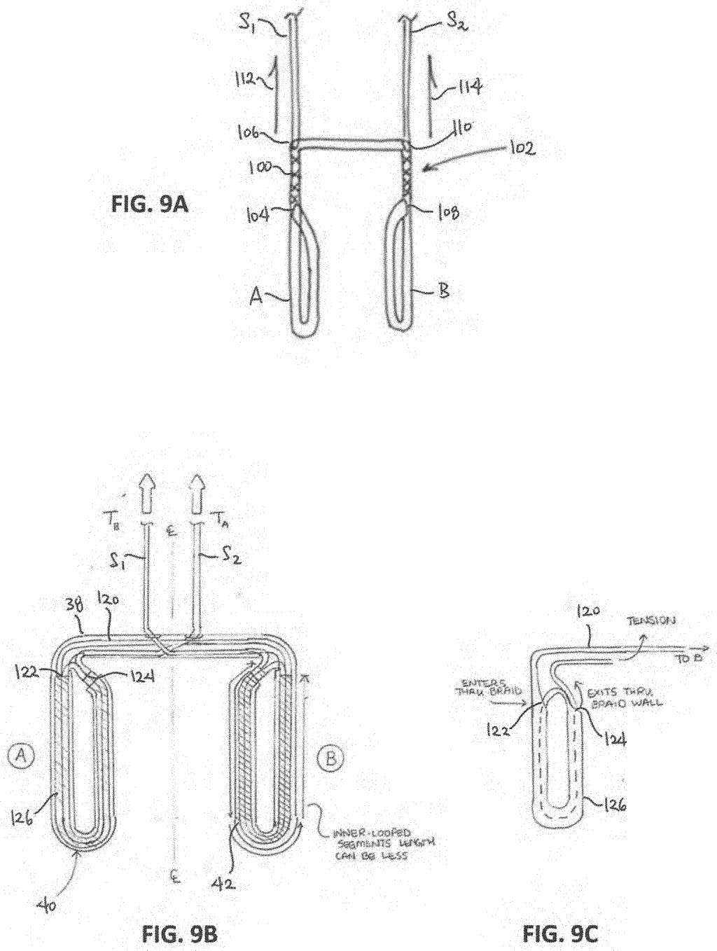

FIG. 9A shows another variation of a knotless suture anchoring system similar to the variation described above. This embodiment may similarly utilize the first terminal end S1 and second terminal end S2 of the suture to be introduced back along a length of the suture but instead of both ends of the suture being introduced along a single bridging region, each terminal end S1, S2 may be introduced back into the suture along its own region. As shown, the first terminal end S1 may form the first loop A but may then be reinserted into the suture along a first point 104 and exit from the suture at second point 106 to form a first spliced section 100. Similarly, the second terminal end S2 may form the second loop B but may then be reinserted into the suture along a first point 108 and exit from the suture at second point 110 to form a second spliced section 102 which is separate from the first spliced section 100. Each of the first points 104,108 may be positioned such that the respective entry points are within the respective openings within the bone and such that the second points 106, 110 are positioned to align with the respective openings within the bone. This may allow the user to tension each terminal end S1, S2 by pulling directly upwards 112, 114 or in a normal direction relative to a surface of the tissue or bone without the need for any knot pushing mechanism. Each leg of the suture staple may be deployed by tensioning the terminal ends S1, S2 either simultaneously or in an alternating fashion to reach the desired compression on the tissue.

FIGS. 9B and 9C show yet another variation of a knotless suture tightening assembly where each end of the suture S1, S2 may be formed into the individual legs 40, 42 and reintroduced into two separate spliced sections. Yet in this variation, each of the legs 40, 42 may be formed by introducing each respective terminal end S1, S2 such that the locking spliced sections occur around a perimeter of each looped section. FIG. 9C shows a detailed side view illustrating how a first length of suture 120 may be initially looped to form the first leg and then introduced back into the suture at a first point 122 such that the suture is then looped again through the interior of the suture to form a first spliced loop section 126 and exited from the interior of the suture at a second point 124. The second loop B may be formed in the same manner as described for the first spliced loop section 126. When pulled, each of the loop sections may cinch down on itself while simultaneously compressing the outer weave or sheath of the suture to form the anchor.

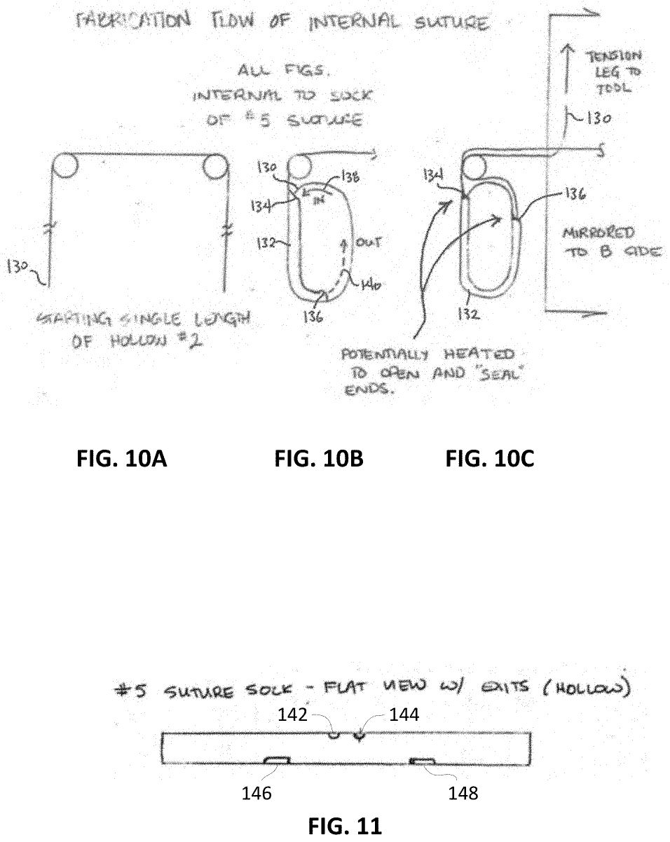

FIGS. 10A to 10C illustrate schematic views of one example for how the spliced portions of suture may be formed. As shown in FIG. 10A, a length of hollow suture (e.g., suture size 2) may be wound upon one or more mandrels and a first terminal end 130 of the suture may be looped at a distance for insertion between the fibers of the suture and back into the lumen of the suture at a first entry point 134 as indicated by the direction 138 in FIG. 10B. The terminal end 130 may be advanced through the suture lumen along a length forming spliced portion 132 until the terminal end 130 is passed through a second exit point 136 as indicated by the direction 140. The terminal end 130 may be passed for tensioning and the process may be repeated for the remaining terminal end of the suture, as indicated in FIG. 10C. The entry point 134 and exit point 136 may be optionally heated to open and then seal the openings.

FIG. 11 shows a side view of one variation of the outer expansile weave of the suture staple in a flattened configuration to illustrate where openings or entry points may be located along its length. As described herein, the suture staple may be formed of a braided tubular structure (e.g., suture size 5) which forms the flexible staple and defines a lumen throughout the length of the staple housing the internal tension sutures. First openings 142, 144 may be formed along a first side of the staple body to accommodate the passage of the first and second terminal ends of the suture for tensioning while second openings 146, 148 may be formed along a second side of the staple body spaced relatively further away from one another than the first openings 142,144 such that the second openings 146, 148 are in proximity to where the legs of the suture staple may be formed to accommodate the passage of the suture into and/or out of the lumen of the staple body.

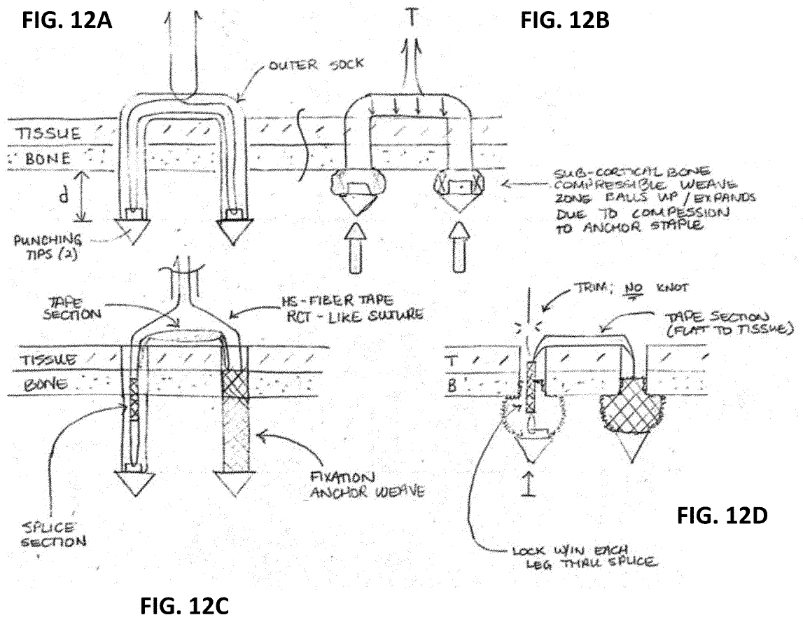

FIGS. 12A and 12B shows another variation of the suture staple deployment in which the tips 20, 22 may be coupled directly to the tensioning suture 150 without the legs of the suture staple 38 being looped. The distal openings of the suture staple 38 may abut against tips 20, 22 such that when suture 150 is tensioned through the openings 152, the tips 20, 22 may be retracted proximally at a distance d, as indicated by the direction of arrows 154 in FIG. 12B, such that the distal portions of the legs 70, 72 may become compressed and expand due to the compression of the tips 20, 22.

FIGS. 12C and 12D show yet another variation in which the tensioning suture may incorporate one or more splice sections along the tensioning suture in one or both members of the suture staple which may abut the tips 20, 22 in a manner similar to FIGS. 12A and 12B. This variation may also incorporate a portion 160 of the suture which may either be configured in a tape configuration (e.g., flattened configuration) or which may incorporate a high-strength fiber tape coupled to the portions of suture. This tape portion 160 may be positioned to extend over the tissue region between the anchoring portions such that the tape portion 160 may lay flat against the tissue surface over the staple bridge to increase the compression surface area on the tissue, as shown in the side views of FIGS. 12C and 12D.

Although the suture staple variations are described with a single leg or looped configuration at either end of the suture staple, other variations of the suture staple may be implemented with any of the other features described herein. For example, other suture staple embodiments may incorporate anchoring configurations in the shape of a toroid, tri-shaped or quad-shaped variations, etc. Moreover, the implant diameters may also be varied at different steps of the procedure.

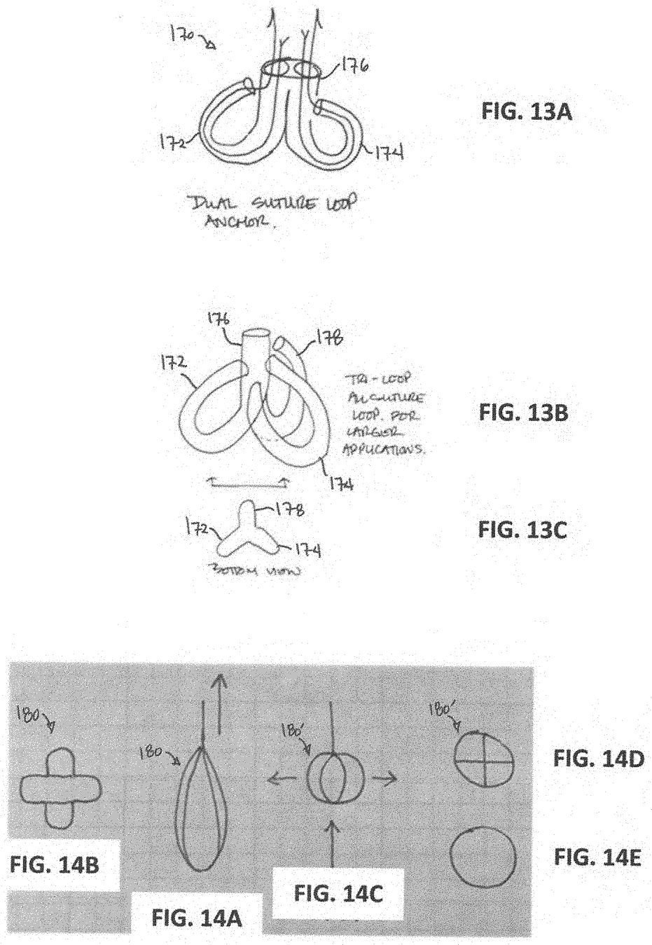

FIG. 13A shows a variation of an anchoring portion of suture staple 170 formed as a dual leg anchor having a first leg 172 and a second leg 174 extending from a common suture staple body 176. FIGS. 13B and 13C show perspective and bottom views of another variation having a tri-loop suture staple embodiment with a third leg 178 in addition to the first 172 and second 174 leg.

FIGS. 14A to 14E show additional variations in which the anchoring portion 180 of the suture staple may be formed in a looped configuration which is reconfigurable upon application of the tensioning force, as shown by anchoring portion 180′ in FIG. 14C. FIG. 14B shows a bottom view of the suture staple anchor 180 in a flattened state while FIGS. 14D and 14E show the bottom view suture staple anchor 180′ in a compressed state.

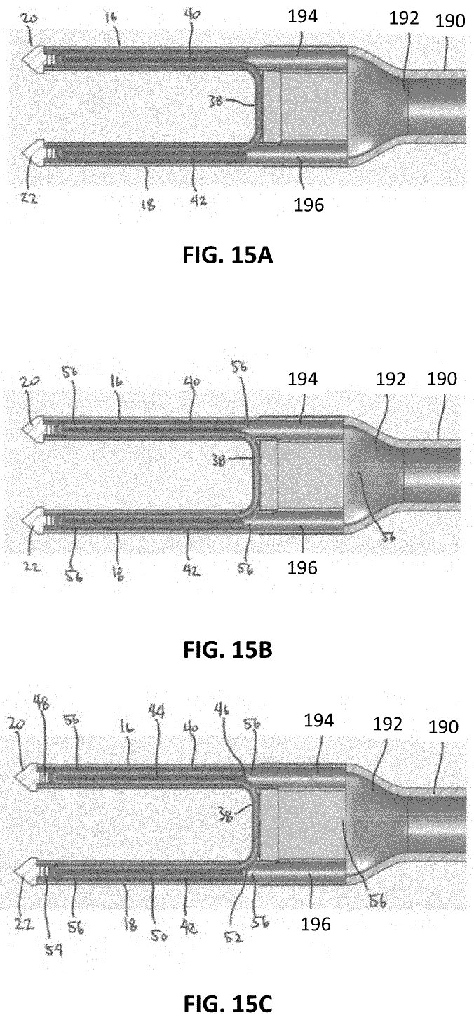

FIGS. 15A to 15C show partial cross-sectional side views of another variation of the anchoring assembly which may be used with any combination of features of any of the suture staple variations described herein. FIG. 15A illustrates the delivery cannulas 16, 18 extending from a delivery shaft 190 which defines a lumen 192 and cannula lumens 194, 196 through which the sutures and tensioning members may pass. The respective detachable tips 20, 22 are shown positioned within the terminal openings of delivery cannulas 16, 18 and the soft staple 38 bridging between each delivery cannula 16, 18 is illustrated with respective legs 40, 42 extending through each cannula 16, 18 with a distal portion bending proximally back through the cannula 16, 18.

FIG. 15B shows the insertion device and staple 38 of FIG. 15A along with the tensioning suture 56 in place. As shown, the tensioning suture 56 may pass through the lumen 192 of delivery shaft 190 and into the body of staple 38. The suture 56 may be routed to pass through the body of staple 38 such that the suture 56 passes through each leg 40, 42 and out through a terminal end of each leg 40, 42 which is bent upon itself and where it may be further passed back into the body of staple 38 and proximally back through delivery shaft 190.

FIG. 15C illustrates the insertion device, staple 38, and tensioning suture 56 of FIG. 15B along with the respective tether 44, 50 which anchors the legs 40, 42 of staple 38 at attachment points 46, 52 to anchoring points 48, 54 of tips 20, 22. As described herein, the tethers 44, 50 help to keep the legs 40, 42 of staple 38 from slipping out of the channels while the tensioning suture 56 is tightened to reconfigure the staple 38 into its anchoring configuration.

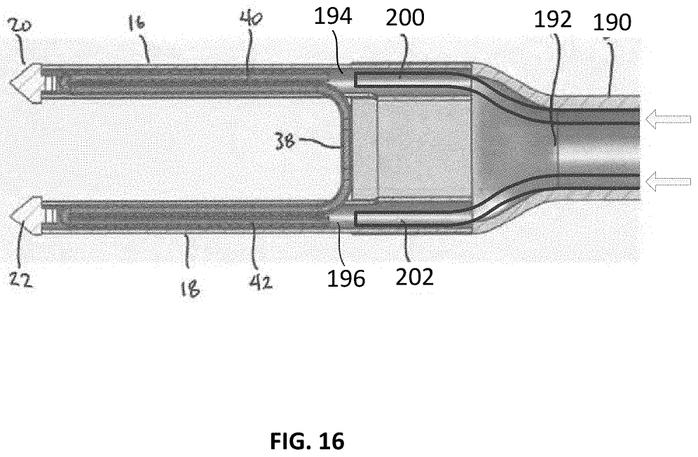

In another variation, the insertion device may optionally incorporate a mechanism to facilitate the deployment of the staple 38 from the insertion device. One example is illustrated in the partial cross-sectional side view of FIG. 16 which illustrates the insertion device having one or more pushing mechanisms such as pushrods 200, 202 which may be advanced distally through respective lumens 194, 196to push the legs 40, 42 of the staple 38 distally out through the terminal end of the delivery cannulas 16, 18 while the body of the staple 38 may slide through the notch or channels defined longitudinally. The pushrods 200, 202 may be configured to be flexible (e.g., nickel-titanium, spring steel, polymer, etc.) and optionally spring loaded to aid in pushing the legs 40, 42 of the staple 38 out distally while the delivery cannulas 16, 18 is retracted from the bone surface.

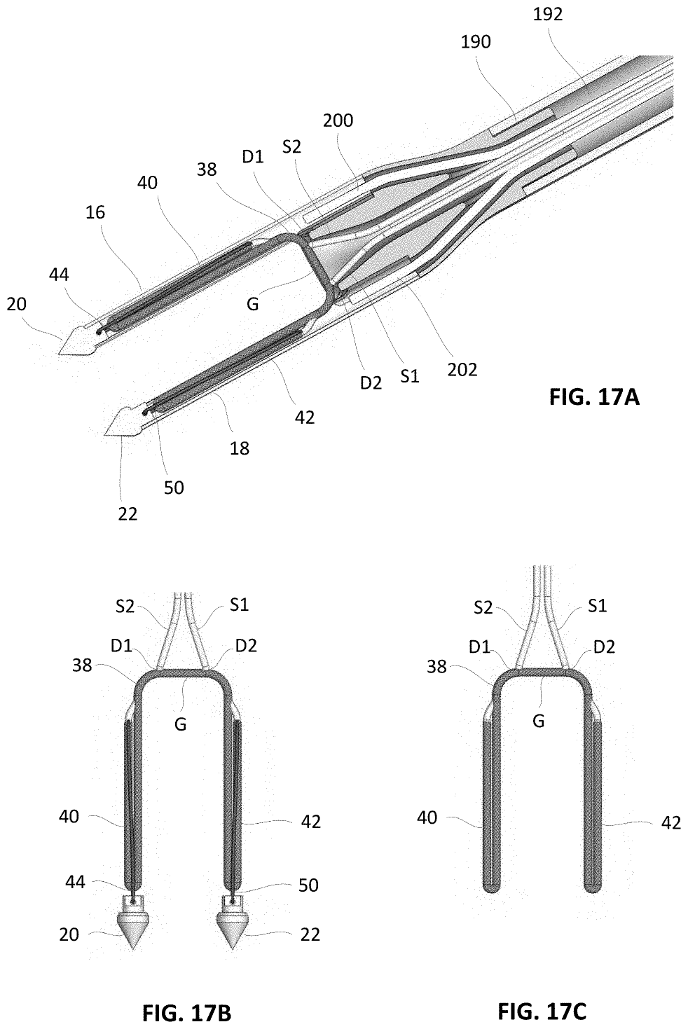

FIGS. 17A to 17C show another variation of the suture staple 38 positioned within the deployment instrument where the suture staple 38 implements the spliced self-locking region G, as described above. The deployment instrument in FIG. 17A incorporates pushrods to assist in holding the staple down into the bone and tissue as the deployment instrument is withdrawn. The pushrods may be released after full insertion into the bone and after released a spring may provide a push force on the staple to counteract friction forces between the staple and the deployment instrument which would act to pull the staple out as the deployment instrument is withdrawn. As shown in FIG. 17A, the ends of the suture staple 38 may be positioned within the respective delivery cannulas 16, 18 with the suture extending through the lumen defined by the suture staple 38 and through the legs 40, 42 to form the suture loops. FIG. 17B illustrates the suture staple 38, tips 20, 22, and tethers 44, 50 coupling the tips 20, 22 to the ends of the suture staple 38 and FIG. 17C illustrates the suture staple 38 with tips 20, 22 and tethers 44, 50 removed for clarity purposes. The suture may be seen extending through suture staple 38 lumen such that the suture extends through the legs 40, 42 to form the respective loops. The ends of the suture S1, S2 may be seen extending from the terminal ends of the legs 40, 42 and back into the suture staple 38 where the spliced self-locking region G may be formed along the suture length within the interior of the suture staple 38.

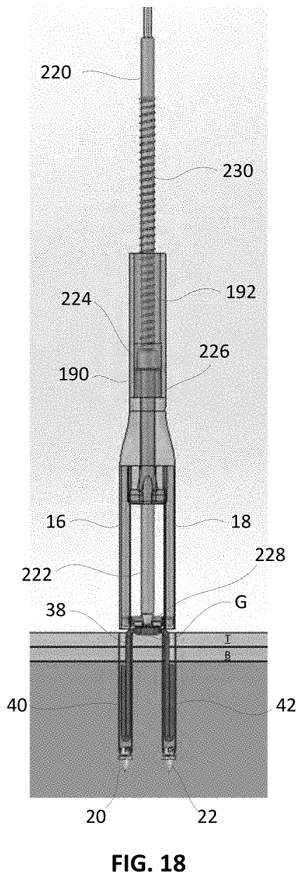

In addition to deploying the tips 20, 22, the deployment instrument may further include a bridge pusher which may be used to prevent the suture staple 38 from being pulled or dragged proximally out of the bone during retraction of the insertion device. FIG. 18 illustrates a partial cross-sectional assembly view of one variation incorporating a bridge pushing mechanism. The deployment instrument may incorporate an elongate shaft 220 slidably positioned within the lumen 192 of the instrument. The shaft 220 may either incorporate a stop mechanism 224 (e.g., an enlarged shoulder or other projection) along the shaft 220 or an additional deployment shaft 222 may be attached or coupled to extend distally from the stop mechanism 224 such that the deployment shaft 222 slidably extends through the lumen 192 and distally between the delivery cannulas 16, 18. A corresponding shoulder 226 may be defined at a terminal end of the lumen 192 for abutting against the stop mechanism 224 during staple deployment. A pushing mechanism 228 (e.g., a transverse member sized to extend between the delivery cannulas 16, 18) may be positioned at a distal end of the deployment shaft 222 and a biasing member 230 may be coupled along the elongate shaft 220 (e.g., a spring positioned over a portion of the shaft 220).

During use, once the delivery cannulas 16, 18 have been inserted into the bone B and the suture staple 38 suitably seated within the bone B for deployment, as described herein, the instrument and cannulas 16, 18 may be withdrawn proximally. While the instrument is withdrawn away from the bone, the elongate shaft 220, stop mechanism 224, and deployment shaft 222 may be advanced distally within the lumen 192 such that the pushing mechanism 228 located at the distal end of the deployment shaft is simultaneously advanced between the delivery cannulas 16, 18 relative to the instrument so that the pushing mechanism 228 remains in contact against the suture staple 38 and the underlying tissue T or bone B. The force applied by the pushing mechanism 228 upon the suture staple 38 during instrument withdrawal may accordingly prevent the suture staple 38 from being pulled or dragged out from the bone B inadvertently. As the delivery cannulas 16, 18 reach the openings in the tissue and the elongate shaft 220 is advanced distally within lumen 192, the stop mechanism 224 may abut the corresponding shoulder 226 such that further advancement of the deployment shaft 222 is halted to prevent the pushing mechanism 228 from extending distally beyond the terminal ends of the delivery cannulas 16, 18. Following removal, the tensioning sutures may freely slide out of the instrument allowing for the suture staple 38 to be further deployed, as described herein.

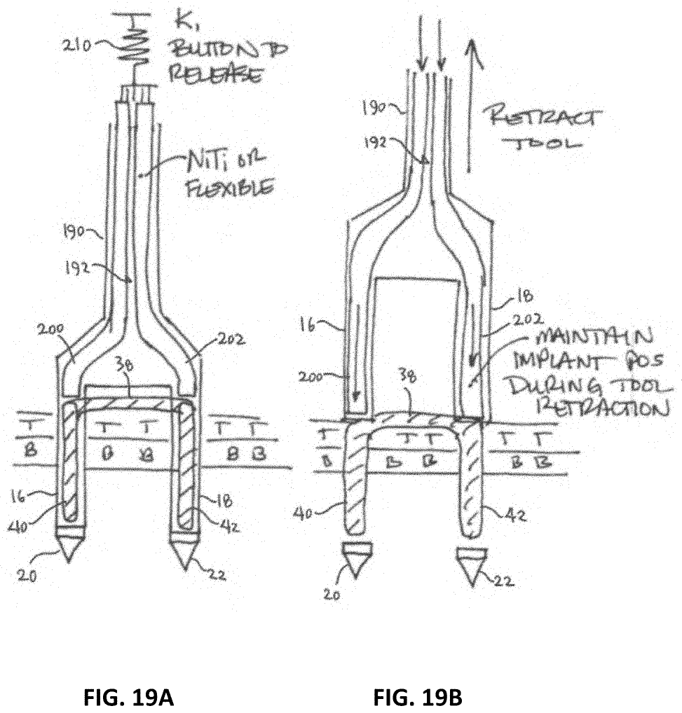

FIGS. 19A and 19B illustrate another variation of the insertion device which may be configured for reducing or eliminating anchor pull back during inserter removal. In this case, two symmetric spring-loaded pushrods 192 may be situated within the device delivery shaft 190 and sit flush to the anchor tine locations. Once the device is fully punched, an actuator (such as a button) can be pressed to release a biasing mechanism 210 having a spring force K1 which is minimal enough that it cannot advance the anchor out of the delivery cannulas 16, 18. As the delivery tool is withdrawn, the pushrods 192 may maintain contact with the implant to prevent the suture staple from withdrawing from the punched position. In additional variations, the pushrods 192 themselves may be hollow and potentially serve as knot pushers for the tensioning phase of the device deployment process. In additional variations a bridging feature may span across the distal end of the pushrods 192 linking the two and uniformly supporting each leg of the anchor and the bridge simultaneously during inserter removal and potentially serve as a single knot pusher for the tensioning phase of the device deployment. Integration of knot pusher functionality enables the user to deploy the implant without the need for removal and insertion of additional instrumentation for the deployment step of the procedure.

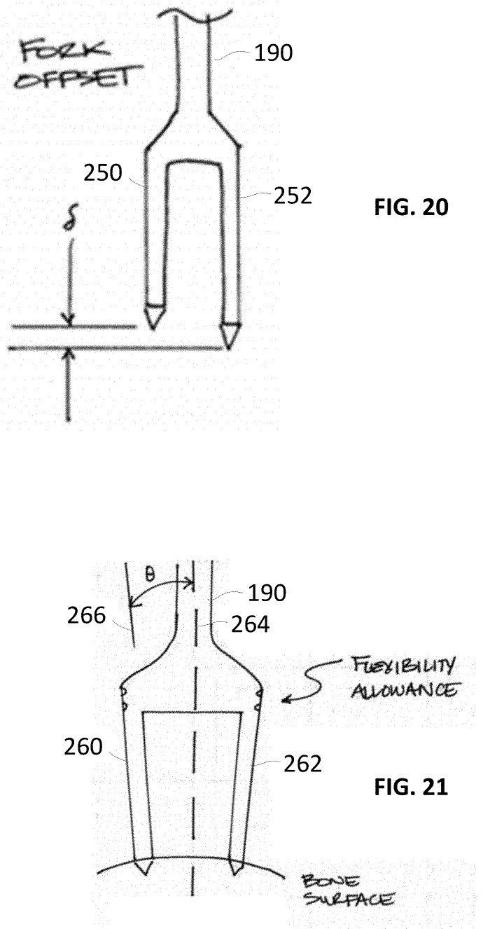

Aside from the delivery actuation mechanisms, the device may also be optionally configured to vary the delivery cannulas to enhance the ability of the device to punch into various uneven patient anatomy such as a curved bone surface. FIG. 20 shows a side view of one variation where a first delivery cannula 250 may be relatively shorter than a second delivery cannula 252 by some nominal distance δ. With a slight offset in the tips, the longer delivery cannula 252 can be initially punched into the bone at a nominal depth equal to the length offset δ before the remaining shorter delivery cannula 250 starts to engage with and punch into bone.

FIG. 21 shows a side view of another variation in which the delivery cannulas 260, 262 may be angled inwardly relative to a longitudinal axis 264 of the insertion device at some offset angle Θ. The delivery cannulas 260, 262 may allow for a certain degree of flexibility to enable the cannulas to be inserted into the bone at such an angle or the cannulas 260, 262 may incorporate some hinge or pivot enabling the cannulas 260, 262 to be inserted at an angled trajectory relative to one another. Such an approach may allow for each anchor portion in the delivery cannulas 260, 262 to take a more nominal depth trajectory relative to the bone surface as if each anchoring portion were individually placed.

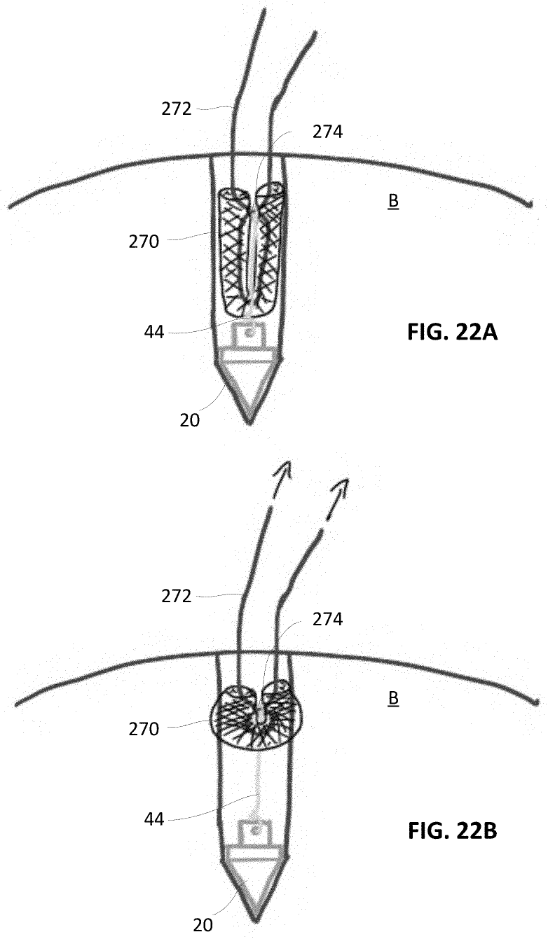

As previously described, another variation of the suture staple 270 may utilize a single cannula to deploy a single leg of the suture staple 270, as illustrated in the side views of FIGS. 22A and 22B. The staple 270 may still utilize a single sharp tip 20 which is coupled to the staple 270 via a tether 44 which similarly retains the staple 270 within the bone B while the suture 272 is tensioned. Rather than conforming the body of the suture staple 270 into a folded configuration, it may instead be utilized in other configurations, such as a straightened configuration or a single looped configuration, etc. which may reconfigure into a coiled or balled up loop configuration. With the staple 270 configured into a single loop configuration, as shown, the tensioning suture 272 may be advanced into a first terminal opening of the staple 270 and passed through the wall of the staple 270 to reenter a lumen of the staple 270. The suture may continue through the internal lumen of the staple 270 where it may again exit through a wall of the staple 270 to reenter through the staple 270 prior to exiting a second terminal opening of the staple 270. The region where the suture 272 exits and reenters the staple 270 may create a cross-over pattern 274. The tether 44 may be secured to the suture 272 at the cross-over 274 location or alternatively secured to one or both ends of the staple 270. In yet another alternative, the tether 44 may be secured to the cross-over 274 location of the suture 272 and a single end of the staple 270.

In this single anchor configuration, the tip 20 may be advanced directly into the bone (e.g., malleted, punched, etc.) for deployment without any need to pre-drill or pre-punch a hole within the bone. The tip 20 may be unattached to the anchor and may act to facilitate the creation of the channel in the bone and/or tissue or the tip 20 may be tethered to the anchor as described herein. As the suture 272 is tensioned, the staple 270 may reconfigure into its coiled and expanded anchoring configuration while initially retained within the bone B via the tether 44 coupled to the tip 20, as shown in FIG. 22B.

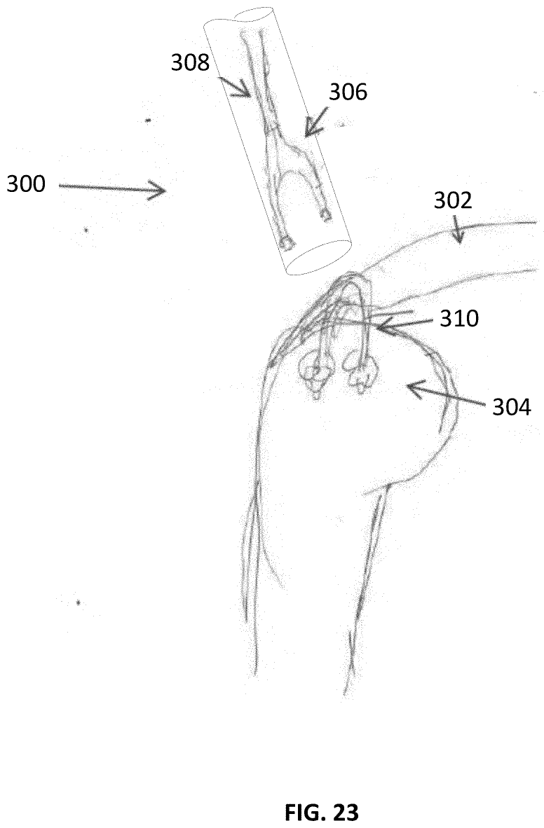

In yet another variation, FIG. 23 illustrates yet another variation of the soft suture staple system 300 used, in this example, to attach partially a torn rotator cuff tissue 302 to the underlying shoulder bone 304. The system 300 may include a variation of the delivery device 308 which may be advanced and deployed through a cannula 306. The distal portion of the delivery device 308 may have two delivery cannulas which extend in parallel (or incorporating any of the variations described herein above). The cannula 306 through which the delivery device 308 is advanced may have a low-profile shape, e.g., an oval cross-section, with the suture staple 310 positioned within and configured to be deployed from the delivery device 308, as described herein. In this example the suture staple 310 is illustrated as having been deployed into a region 304 along the shoulder bone. The deployment instrument 308 may be initially inserted through an incision near, e.g., the patient's lateral acromion and extended through the deltoid muscles and bursal tissue.

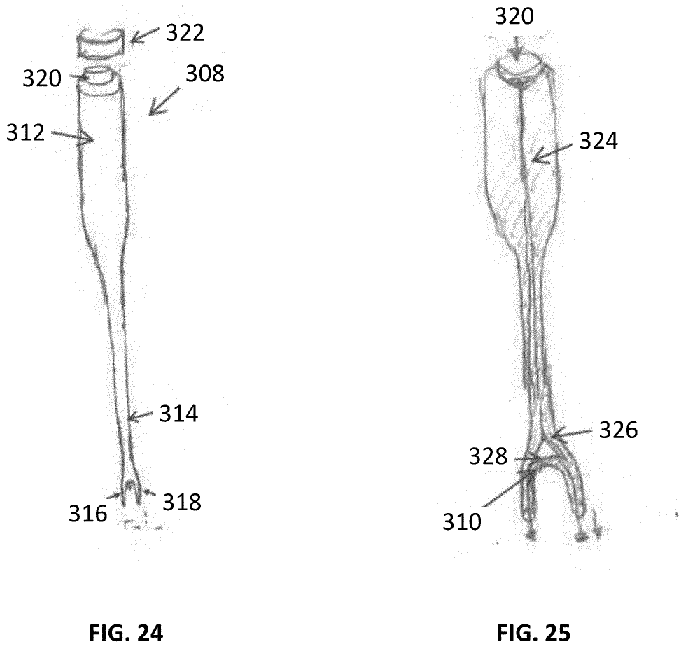

As shown in FIG. 24, a side view of the delivery device 308 illustrates a variation where the device 308 may have a tubular housing having a slightly enlarged proximal portion 312 designed to function as a handle, and a narrower distal portion 314. The distal end of the distal portion 314 may have the first delivery cannula 316 and second delivery cannula 318 project from the distal portion. The proximal end of the proximal portion 312 may include one or more actuation features such as a button 320 which may be covered by a protective cap 322 which allows for the delivery device 308 to be pushed or pounded with, e.g., a mallet or other instrument, when driving the suture staple 310 into region of bone, as described herein.

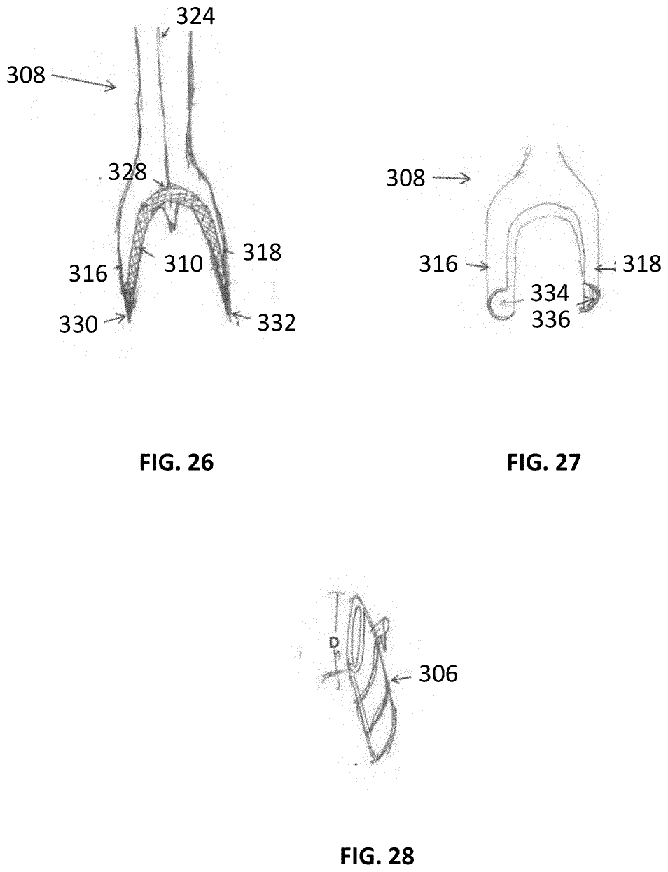

The actuation button 320 may be coupled or connected to pushrods (as described herein) or to a a driving rod 324 which may extend through the interior of the device 308 and to a distal end 326 that, prior to deployment, may reside above the proximal surface 328 of the suture staple 310, as shown in partial cross-sectional side view of FIG. 25. When the button 320 is actuated, the pushrods of driving rod 324 may be urged distally within the device 308 and cause its distal end 326 to contact the proximal surface 328 of suture staple 310, as shown in detail side view of FIG. 26.

The suture staple 310 may be positioned within the device 308 where its terminal portions may extend within each respective delivery cannula 316, 318 such that its suture bridge extends between. As described above, the tips 330, 332 may be positioned at each distal opening of each respective delivery cannula 316, 318 for coupling to the suture staple 308. In this variation, the dimensions of the suture staple 310 have been selected to normally create a press fit between the suture staple 310 and the delivery cannulas 316, 318, holding the staple 310 in place. However, once the tips 330, 332 of the suture staple 310 have been driven into bone, the anchoring forces on the tips 330, 332 may overcome the press fit, allowing the delivery device 308 to be withdrawn and separated from the suture staple 310.

As shown in FIG. 27, the delivery cannulas 316, 318 are illustrated with the suture staple 310 removed for clarity purposes. As shown, the inner side walls 334, 336 of the delivery cannulas 316, 318 may be configured to be concave so as to allow the delivery cannulas 316, 318 to fully engage and surround the distal surface of the suture staple 310 to enhance the strength of the press fit while allowing for the distal deployment of the suture staple 310 into the tissue.

FIG. 28 illustrates a portion of the delivery cannula 306 to illustrate how the cannula 306 may be configured to have an oval shape so as to minimize the incision width through the skin and underlying tissue when inserted into proximity to the region of the bone and tissue for treatment. The oval shape may also allow for the passage of the delivery device 308 through, for example, the deltoid and subacromial tissue while limiting water leakage and tissue expansion during a repair procedure. In this example, the diameter D of the cannula 306 at its major axis may be about, e.g., 12 mm, and at its transverse minor axis may be about, e.g., 10 mm, in order to provide sufficient clearance for a delivery device 308. While an oval shape is shown, other configurations may also be utilized for the delivery cannula 306, e.g., circular, rectangular, etc., and other dimensions may be utilized.

While the delivery cannula 306 is shown here, the cannula 306 may be used in any combination with any of the other delivery device embodiments and suture staple variations described herein.

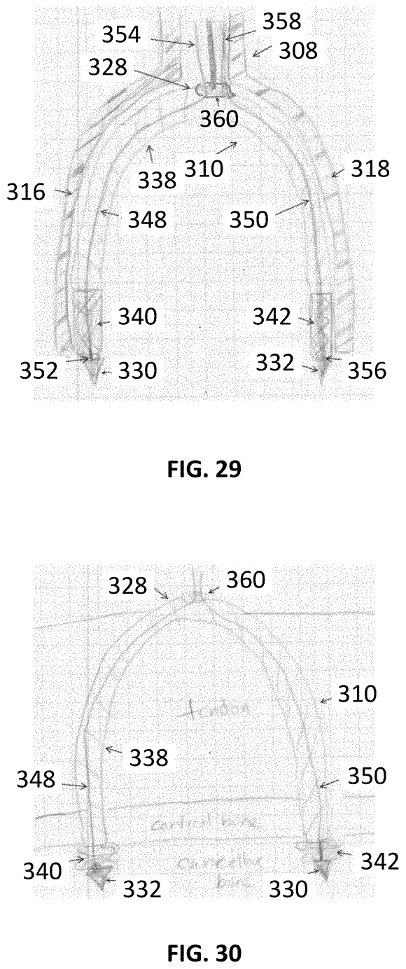

FIG. 29 shows a partial cross-sectional side view of another variation of the soft suture staple 310 positioned within the delivery device 308 for deployment. The suture staple 310 may be comprised of any of the variations as described herein while in this variation, the suture staple 310 may include a coreless multifilament woven suture formed of a material such as poly (ethylene terephthalate) formed into a tubular configuration. The body 338 of the suture staple 310 may be formed as a water-resistant portion and the distal portions 340, 342 of the suture staple 310 may each be formed as water-absorbent portions. In this manner, the water-resistant body 338 of the suture staple 310 may extend between each of the delivery cannulas 316, 318 while the water-absorbent portions 340, 342 of each distal end may be positioned within the distal ends of each respective delivery cannula 316, 318.

The body portion 338 may be stiffened, e.g., with a water-resistant polymeric coating, while the absorbent portions 340, 342 may be formed, for example, of a suture tape comprising several braided strands of uncoated fiber.

As described above, the respective tips 330, 332 may be positioned within the terminal openings of each respective delivery cannula 316, 318 and may be formed of any of the tip materials described hereinabove. The tensioning sutures 348, 350 may extend through the lumen defined by the suture staple 310 such that the first tensioning suture 348 may be secured to the first tip 330 at anchor 352 along the first tip 330 and the second tensioning suture 350 may be secured to the second tip 332 at anchor 356 along the second tip 332 such that each tensioning suture 348, 350 may extend through the suture staple lumen and pass through a proximal opening 328 along the body 338 of suture staple 310 such that the respective suture ends 354, 358 each pass through the opening 328 and extend proximally through the device 308.

The tensioning sutures 348, 350 may optionally extend through a locking device or ratcheting mechanism 360 (e.g., zip tie insert, inserts, etc.) positioned along the proximal opening 328 along the body 338. Alternatively, the tensioning sutures 348, 350 may be provided with projections (e.g., barbs, shoulders, etc.) that allow for incremental movement in one direction during tensioning of the sutures 348, 350.

When the suture staple 310 is deployed, the tips 330, 332 may be driven or introduced directly into the tissue and underlying bone. In one example, after a partial rotator cuff tear has been identified using diagnostic arthroscopy inside the joint capsule, a shaver may be inserted through an anterior portal and used to lightly debride frayed tissues and greater tuberosity bone. A spinal needle may be used near the edge of the lateral acromion to find an approximate trajectory to the bone, and an incision (e.g., 1.5 cm) may be made with a scalpel to allow placement of the oval cannula 306. Once the deltoid muscles and bursal tissues have been penetrated, an inner obturator is withdrawn, and the cannula 306 may be rotated to clear most of the surrounding tissue. The delivery device 308 may then be placed into the cannula 306 to penetrate the rotator cuff at the appropriate angle and location for proper fixation and the delivery device 308 may be advanced, e.g., malleted, until the delivery cannulas 316, 318 and ends of the suture staple 310 are buried in the cancellous bone, compressing the rotator cuff tissue against the cortical bone. The end cap 322 may be removed from the proximal end of the delivery device 308 and the device actuated to deploy the suture staple 310 from the delivery cannulas 316, 318 while the delivery device 308 may be slowly retracted leaving the soft suture staple 310 in the bone as shown in FIG. 30.

Once the suture staple 310 has been placed within the bone, a knot pusher may be optionally used to press down on the proximal surface 328 of the suture staple 310, while pulling on the inner core tensioning sutures 348, 350. The downward pressure on the staple 310 combined with the proximally directed tension on the tensioning sutures 348, 350 may cause the absorbent portions 340, 342 of the suture staple 310 to compress longitudinally between the tips 330, 332 and the body portion 338 such that the absorbent portions 340, 342 may reconfigure and collapse such that they expand laterally into an anchoring configuration which optimizes frictional engagement with the cancellous bone. The tensioning sutures 348, 350 can then be locked in place using, e.g., locking mechanism 360 and the suture tails 354, 358 may be cut, e.g., 2 mm above the staple surface.

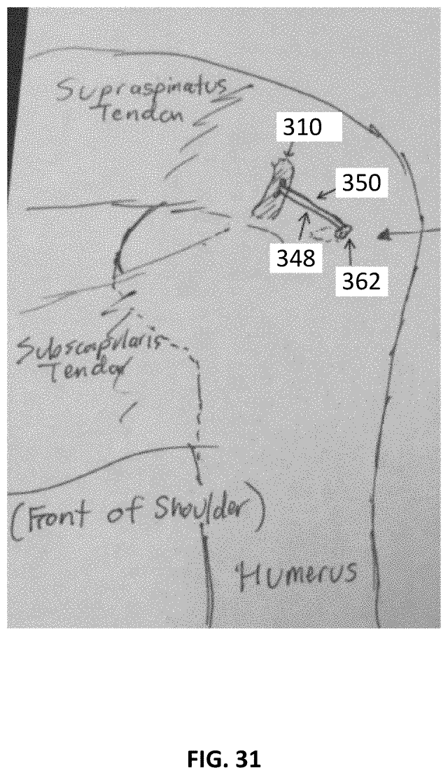

If desired, the repair can be further secured by optionally placing the tensioning sutures 348, 350 into a knotless anchor 362 and placed laterally on the greater tuberosity to make a T-type repair pattern, as shown in FIG. 31.

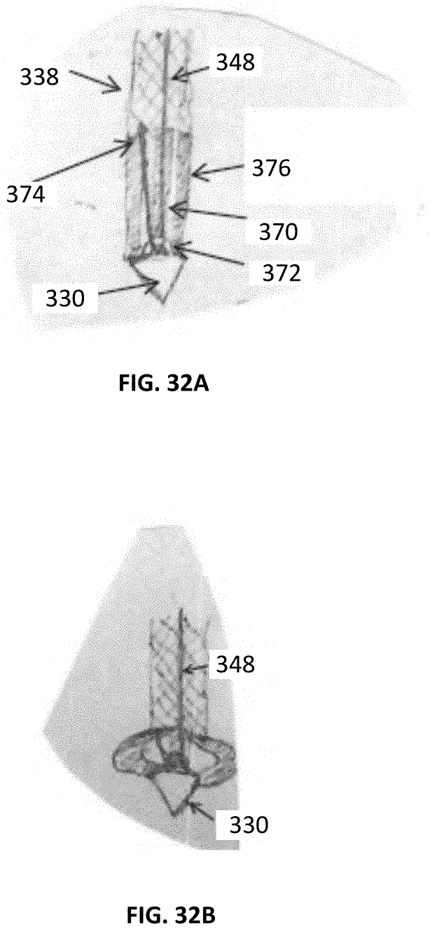

FIGS. 32A and 32B show side views of another variation of the suture staple in a single exemplary leg of the suture staple. In this embodiment, the distal end 370 of the respective tensioning suture 348 may be passed through the anchor 372 on the proximal surface of tip 330 and secured to a portion along a distal edge 374 of the water-resistant body portion 338 of the suture staple 310. When the tensioning suture 348 is tensioned, the distance between the tip 330 and the distal edge 374 of the body portion 338 of the suture staple may be decreased, causing the strands of the distal end 376 to compress longitudinally and expand laterally as shown in FIG. 32B, allowing for anchoring engagement with the bone.

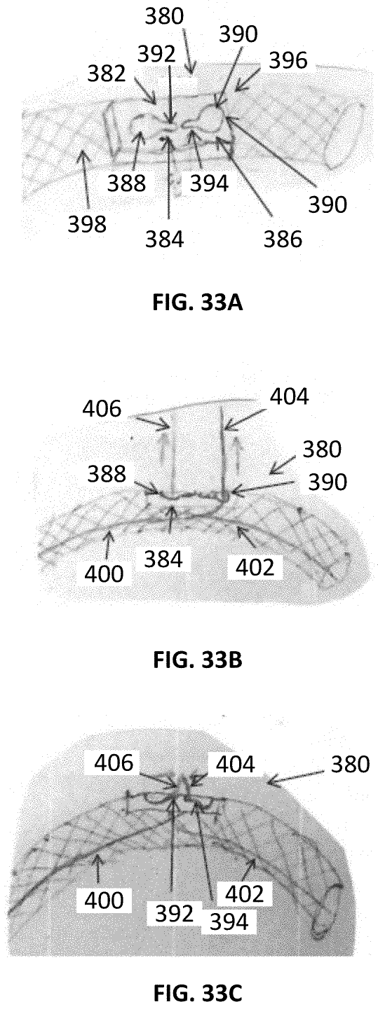

FIGS. 33A to 33C show one variation of the locking mechanism 380 for optionally securing the tensioning sutures 400, 402 once the absorbent portions 340, 342 of the suture staple 310 have expanded into engagement with the cancellous bone. The locking mechanism 380 may include an insert 382 disposed on the proximal surface 396 of the suture staple 398. A pair of keyhole slots 384, 386 may be formed in the proximal surface of the insert 382. As the proximal tail 404 of the first tensioning suture 400 crosses over the proximal tale 406 of the second tensioning suture 402 and passes through the enlarged portion 390 of second keyhole slot 386, while the proximal tail 406 of the second tensioning suture 402 passes through the enlarged portion 388 of the first keyhole slot 384, as shown in FIG. 33B.

When sufficient tension has been applied to tensioning sutures 400, 402, the tensioning sutures 400, 402 may be urged towards one another to force the proximal tails 404, 406 into the narrowed portions 392, 394 of their respective keyhole slots 384, 386, as shown in FIG. 33C. Once the proximal tails 404, 406 are tightly secured in the narrowed portions 392, 394 of the slots 384, 386, they may be cut to a length of, e.g., about 2 mm, above the staple surface.

Use of the soft suture staple system 300 (or any of the variations described herein) are not limited to any particular procedure (e.g., repairing PASTA tears) but may be used in any number of procedures such as repair of mildly retracted full thickness rotator cuff tears and in remplis sage procedures for Hills Sachs lesions of the humeral head, etc.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A tissue anchor assembly, comprising:
an insertion device having a first delivery cannula and a second delivery cannula where the first and second delivery cannulas project distally while defining a notch or opening along each delivery cannula;
a first tip removably positioned within a first terminal opening of the first delivery cannula and a second tip removably positioned within a second terminal opening of the second delivery cannula, wherein each of the first and second tips is configured to pierce a tissue region;
a flexible staple having a body portion and a first leg portion at a first end of the body portion and a second leg portion at a second end of the body portion, wherein each of the first and second leg portions is configured to fold proximally upon itself within a respective first and second delivery cannula such that the body portion extends between the first and second delivery cannula; and
a tensioning suture passing through a lumen defined by the flexible staple such that the suture passes through the first and second leg portions of the flexible staple and extends back into the lumen to form a loop in each leg portion of the flexible staple such that application of a tensioning force upon a proximal portion of the tensioning suture urges the first leg and second leg to each reconfigure into a collapsed, anchoring configuration.

2. The assembly of claim 1 wherein each end of the tensioning suture passes through its own core at a spliced portion along the suture within which ends of the suture overlap each other and extend in opposing directions such that the collapsed, anchoring configuration is self-locking.

3. The assembly of claim 1 wherein the first delivery cannula and second delivery cannula are configured to have a column strength sufficient for advancement directly into soft tissue or bone.

4. The assembly of claim 1 further comprising a first tether coupling the first tip to the first leg and a second tether coupling the second tip to the second leg.

5. The assembly of claim 4 wherein the first and second tethers comprise a suture length.

6. The assembly of claim 4 wherein the first and second tethers comprise members extending from each respective first and second tips.

7. The assembly of claim 1 wherein the first and second tips each comprise an anchoring location for coupling to a respective first leg and second leg.

8. The assembly of claim 1 wherein the first and second tips each comprise a piercing tip which is offset relative to a longitudinal axis of each tip.

9. The assembly of claim 1 wherein the first and second tips each define an outer surface which is configured for enhancing frictional grip.

10. The assembly of claim 1 wherein the first and second tips each define an outer diameter which is relatively larger than an outer diameter of a respective delivery cannula.

11. The assembly of claim 1 wherein the first and second tips each define a cutout portion.

12. The assembly of claim 1 wherein the flexible staple comprises a braided tubular structure.

13. The assembly of claim 1 wherein the tensioning suture passes back into the body portion at a proximal end of each leg portion.

14. The assembly of claim 2 wherein the spliced portion comprises a hollow portion of the tensioning suture defined via an entry point and an exit point with both ends of the suture passing through the splice in opposing directions.

15. The assembly of claim 14 wherein the spliced portion is configured to collapse when tensioned such that a locking configuration of the tensioning suture is maintained.

16. A method of securing a tissue anchor, comprising:
advancing a first delivery cannula and a second delivery cannula located adjacent to the first delivery cannula directly into a tissue region such that a first channel and a second channel are formed respectively within the tissue region;
retracting the first delivery cannula and the second delivery cannula from the first channel and the second channel such that a first tip is detached from the first delivery cannula and remains within the first channel and a second tip is detached from the second delivery cannula and remains within the second channel;
deploying a first leg portion from the first cannula and a second leg portion from the second cannula such that the first leg portion and second leg portion remain connected to one another via a body portion of a flexible staple, wherein the first leg portion is folded proximally upon itself within the first channel and the second leg portion is folded proximally upon itself within the second channel; and
applying a force to a tensioning suture passing through the body portion, the first leg, and the second leg such that the first leg portion and the second leg portion are each urged to reconfigure into a collapsed, anchoring configuration within the respective first and second channels.

17. The method of claim 16 wherein the suture extends back into the body portion to form a spliced portion along the suture within which ends of the suture extend in opposing directions such that the collapsed, anchoring configuration is self-locking.

18. The method of claim 16 wherein the first leg portion is coupled to the first tip within the first channel via a first tether and the second leg portion is coupled to the second tip within the second channel via a second tether.

19. The method of claim 16 wherein advancing the first delivery cannula and the second delivery cannula directly into the tissue region comprises advancing the first and second delivery cannulas into a bone region.

20. The method of claim 16 wherein deploying the first leg portion from the first cannula and the second leg portion from the second cannula comprises passing the body portion through a notch or opening defined along a length of the first and second cannulas.

21. The method of claim 16 wherein deploying the first leg portion from the first cannula and the second leg portion from the second cannula comprises ejecting the leg portions via a pushrod advanced distally.

22. The method of claim 16 wherein the flexible staple comprises a braided tubular structure.

23. The method of claim 16 wherein applying the force to the tensioning suture comprises passing the tensioning suture through a hollow portion of the tensioning suture via an entry point and an exit point.

24. The assembly of claim 23 wherein the at least one spliced portion is configured to collapse when tensioned such that a locking configuration of the tensioning suture is maintained.

25. A tissue anchor assembly, comprising:
a first tip and a second tip each configured to pierce a tissue region;
a flexible staple having a body portion and a first leg portion at a first end of the body portion and a second leg portion at a second end of the body portion, wherein each of the first and second leg portions is configured to fold proximally upon itself;
a first tether coupling the first tip to the first leg and a second tether coupling the second tip to the second leg; and
a tensioning suture passing through a lumen defined by the flexible staple such that the suture passes through the first and second leg portions of the flexible staple and extends back into the lumen such that application of a tensioning force upon the tensioning suture urges the first leg and second leg to each reconfigure into a collapsed, anchoring configuration.

26. The assembly of claim 25 wherein the tensioning suture extends back into the lumen to form a spliced portion along the suture within which ends of the suture extend in opposing directions such that the collapsed, anchoring configuration is self-locking.

27. The assembly of claim 25 further comprising an insertion device having a first delivery cannula and a second delivery cannula adjacent to the first delivery cannula such that the first and second delivery cannulas project distally while defining a notch or opening along each delivery cannula.

28. The assembly of claim 27 wherein the first delivery cannula and second delivery cannula are offset in length relative to one another.

29. The assembly of claim 27 wherein the first delivery cannula and second delivery cannula are offset in angle relative to a longitudinal axis of the insertion device.

30. The assembly of claim 27 wherein the first tip is removably positioned within a first terminal opening of the first delivery cannula and a second tip is removably positioned within a second terminal opening of the second delivery cannula.

31. The assembly of claim 25 wherein the tensioning suture comprises at least one spliced portion where the tensioning suture is passed through a hollow portion of the tensioning suture via an entry point and an exit point.

32. The assembly of claim 31 wherein the at least one spliced portion is configured to collapse when tensioned such that a locking configuration of the tensioning suture is maintained.

33. A tissue anchor assembly, comprising:
an insertion device having a first delivery cannula and a second delivery cannula adjacent to the first delivery cannula such that the first and second delivery cannulas project distally while defining a notch or opening along each delivery cannula;
a first tip removably positioned within a first terminal opening of the first delivery cannula and a second tip removably positioned within a second terminal opening of the second delivery cannula, wherein each of the first and second tips is configured to pierce a tissue region;
a flexible staple having a body portion and a first leg portion at a first end of the body portion and a second leg portion at a second end of the body portion; and
a tensioning suture passing through a lumen defined by the flexible staple such that the suture passes through the first and second leg portions of the flexible staple and extends back into the lumen such that application of a tensioning force upon a proximal portion of the tensioning suture urges the first leg and second leg to each reconfigure into a self-locking collapsed, intussuscepted configuration.

34. The assembly of claim 33 wherein the tensioning suture forms a spliced portion along the suture within which ends of the suture extend in opposing directions such that the collapsed, intussuscepted configuration is self-locking.

35. The assembly of claim 33 wherein the first delivery cannula and second delivery cannula are configured to have a column strength sufficient for advancement directly into soft tissue or bone.

36. The assembly of claim 33 wherein the first delivery cannula and second delivery cannula are offset in length relative to one another.

37. The assembly of claim 33 wherein the first delivery cannula and second delivery cannula are offset in angle relative to a longitudinal axis of the insertion device.

38. The assembly of claim 33 wherein the first and second tips each comprise an anchoring location for coupling to a respective first leg and second leg.

39. The assembly of claim 33 wherein the first and second tips each comprise a piercing tip which is offset relative to a longitudinal axis of each tip.

40. The assembly of claim 33 wherein the first and second tips each define an outer surface which is roughened for enhancing frictional grip.

41. The assembly of claim 33 wherein the first and second tips each define a cutout portion.

42. The assembly of claim 33 wherein the first and second tips each comprise one or more radially extending arm members configured to expand upon tensioning of the first leg and second leg into the collapsed, anchoring configuration.

43. The assembly of claim 33 wherein the flexible staple comprises a braided tubular structure.

44. The assembly of claim 33 wherein the tensioning suture passes through a lumen defined through a length of the staple.

45. The assembly of claim 33 further comprising a first tether coupling the first tip to the first leg and a second tether coupling the second tip to the second leg.

46. The assembly of claim 45 wherein the first and second tethers comprise a suture length.

47. The assembly of claim 45 wherein a distal end of the first leg is coupled to the first tip and a distal end of the second leg is coupled to the second tip.

48. The assembly of claim 34 wherein the tensioning suture comprises at least one spliced portion where the tensioning suture is passed through a hollow portion of the tensioning suture via an entry point and an exit point.

49. The assembly of claim 48 wherein the at least one spliced portion is configured to collapse when tensioned such that a locking configuration of the tensioning suture is maintained.

50. A method of securing a tissue anchor, comprising:
advancing a first delivery cannula and a second delivery cannula located adjacent to the first delivery cannula directly into a tissue region such that a first channel and a second channel are formed respectively within the tissue region;
retracting the first delivery cannula and the second delivery cannula from the first channel and the second channel such that a first tip is detached from the first delivery cannula and remains within the first channel and a second tip is detached from the second delivery cannula and remains within the second channel;
deploying a first leg portion from the first cannula and a second leg portion from the second cannula such that the first leg portion and second leg portion remain connected to one another via a body portion of a flexible staple; and
applying a force to a tensioning suture passing through the body portion, the first leg, and the second leg such that the first leg portion and the second leg portion are each urged to reconfigure into a collapsed, intussuscepted configuration within the respective first and second channels.

51. The method of claim 50 wherein the suture extends back into the body portion to form a spliced portion along the suture within which ends of the suture extend in opposing directions such that the collapsed, intussuscepted configuration is self-locking.

52. The method of claim 50 wherein advancing the first delivery cannula and the second delivery cannula directly into the tissue region comprises advancing the first and second delivery cannulas into a bone region

53. The method of claim 50 wherein deploying the first leg portion from the first cannula and the second leg portion from the second cannula comprises passing the body portion through a notch or opening defined along a length of the first and second cannulas.

54. The method of claim 50 wherein the first leg portion is coupled to the first tip within the first channel via a first tether and the second leg portion is coupled to the second tip within the second channel via a second tether.

55. The method of claim 50 wherein the flexible staple comprises a braided tubular structure.

56. The method of claim 50 wherein applying the force to the tensioning suture comprises passing the tensioning suture through a hollow portion of the tensioning suture via an entry point and an exit point.

57. The assembly of claim 50 wherein the at least one spliced portion is configured to collapse when tensioned such that a locking configuration of the tensioning suture is maintained.

58. A tissue anchor assembly, comprising:
an insertion device having a delivery cannula which projects distally;
a tip removably positioned within a terminal opening of the delivery cannula, wherein the tip is configured to pierce a tissue region;
a flexible anchor having a leg portion wherein the leg portion is configured to fold upon itself within the delivery cannula;
a tether coupling the tip to the flexible anchor; and
a tensioning suture passing through a lumen defined by the flexible anchor such that the suture passes through the flexible anchor to form a cross-over pattern whereby ends of the suture extend through terminal openings of the flexible anchor in opposing directions such that application of a tensioning force upon the tensioning suture urges the flexible anchor to reconfigure into a collapsed, anchoring configuration.

59. The assembly of claim 58 wherein the delivery cannula is configured to have a column strength sufficient for advancement directly into soft tissue or bone.

60. The assembly of claim 58 wherein the tip defines an outer surface which is configured for enhancing frictional grip.

61. The assembly of claim 58 wherein the tip defines an outer an outer diameter which is relatively larger than an outer diameter of the delivery cannula.

62. The assembly of claim 58 wherein the flexible staple comprises a braided tubular structure.
